(19) United States
(12) Patent Application Publication
(10) Pub. No.: US 2026/0151064 A1
(43) Pub. Date: Jun. 4, 2026

(54) Storage Medium, Information Processing Method, and Information Processing Device

(71) Applicant: The University of Osaka, Suita-shi, Osaka (JP)

(72) Inventors: Gajanan Subhash Revankar, Suita-shi, Osaka (JP); Hideki Mochizuki, Suita-shi, Osaka (JP); Ken Nakata, Suita-shi, Osaka (JP)

(21) Appl. No.: 19/126,376

(22) PCT Filed: Oct. 31, 2023
(86) PCT No.: PCT/JP2023/039327

§ 371 Date: May 1, 2025

(30) Foreign Application Priority Data
Nov. 9, 2022 (JP) 2022-179766

(51) Int. Cl. A61B 5/16; A61B 5/00; G06T 7/00; G06T 7/70; G10L 25/66; G16H 10/20; G16H 50/30

(52) U.S. Cl. CPC A61B 5/165; A61B 5/163; A61B 5/4803; A61B 5/7267; A61B 5/7275; G06T 7/0012; G06T 7/70; G10L 25/66; G16H 10/20; G16H 50/30; G06T 2207/20081; G06T 2207/30041; G06T 2207/30201

(57) ABSTRACT

Provided is a program, etc. capable of improving accuracy of a test result obtained by a pareidolia test. A computer outputs a test image used in a pareidolia test. In addition, the computer acquires information related to a response of a subject to the output test image. Then, the computer computes a risk score related to a neuropsychiatric disorder based on the acquired information related to the response of the subject.

### REFERENCE TO RELATED APPLICATIONS

This application is the national phase under 35 U.S.C. § 371 of PCT International Application No. PCT/JP2023/039327 which has an International filing date of Oct. 31, 2023 and designated the United States of America.

### BACKGROUND ART

The number of patients having neuropsychiatric disorders such as Parkinson's disease, Lewy body dementia, Alzheimer's disease, and schizophrenia is increasing, and is expected to continue to increase in the future. Patients having such neuropsychiatric disorders exhibit various symptoms, such as mood disorders, impulsivity, hallucinations, and visual hallucinations (illusions and optical illusions), and since these symptoms significantly impair the quality of life of patients, it is important to provide appropriate treatment early.

For evaluation of visual hallucinations, NPI (Neuropsychiatric Inventory) scores, MDS-UPDRS (Movement Disorder Society Unified Parkinson's Disease Rating Scale) scores, etc. have been used. In addition, for diagnosis of visual hallucinations, MIBG (Meta Iodobenzyl Guanidine) myocardial scintigraphy tests, Dat scan tests, EEG (Electro Encephalo Graphy), brain MRI (Magnetic Resonance Imaging) tests, etc. have been used. However, since it is difficult to appropriately evaluate and diagnose visual hallucinations using these tests, and imaging devices used in these tests are expensive, the percentage of medical institutions adopting the imaging devices is low, and the number of medical institutions that can perform the tests is limited.

Japanese Published Patent Publication No. 2020-537579 proposes technology that predicts and diagnoses neurological disorders such as Parkinson's disease and stroke using a trained diagnostic system from at least one of a video record of a patient and an audio record of spoken voice of the patient.

### SUMMARY

The visual hallucinations seen in patients having neuropsychiatric disorders are called “pareidolia”, a condition in which real objects are mistakenly perceived, and a pareidolia test is conducted to examine the degree of pareidolia symptoms. However, the pareidolia test is considered to have high specificity for pareidolia but low sensitivity, and there is demand for improving accuracy of a test result obtained by the pareidolia test. The technology disclosed in Japanese Published Patent Publication No. 2020-537579 predicts the presence and severity of a neurological disorder from at least one of a video record and an audio record of a patient, and does not perform predictive diagnosis related to pareidolia.

An object of the disclosure is to provide a storage medium, etc. capable of improving accuracy of a test result obtained by a pareidolia test.

A non-transitory computer-readable storage medium according to an aspect of the disclosure stores a program causing a computer to execute processing of outputting a test image used in a pareidolia test, acquiring information related to a response of a subject to the test image, and computing a risk score related to a neuropsychiatric disorder based on the information related to the response of the subject.

According to an aspect of the disclosure, it is possible to improve accuracy of a test result obtained by a pareidolia test.

The above and further objects and features will more fully be apparent from the following detailed description with accompanying drawings.

### BRIEF DESCRIPTION OF DRAWINGS

FIG. 1 is an explanatory diagram illustrating a configuration example of an information processing system.

FIG. 2 is a block diagram illustrating a configuration example of a server and a user terminal.

FIG. 3A is an explanatory diagram illustrating an overview of a noise image generation model.

FIG. 3B is an explanatory diagram illustrating an overview of a facial image generation model.

FIG. 4A is an explanatory diagram illustrating an overview of the facial image generation model.

FIG. 4B is an explanatory diagram illustrating an overview of the facial image generation model.

FIG. 5A is an explanatory diagram illustrating an example of a pareidolia test image.

FIG. 5B is an explanatory diagram illustrating an example of a pareidolia test image.

FIG. 6A is an explanatory diagram illustrating an overview of an eye gaze tracking score computation model.

FIG. 6B is an explanatory diagram illustrating an overview of an utterance tracking score computation model.

FIG. 7A is an explanatory diagram of an eye gaze tracking score.

FIG. 7B is an explanatory diagram of an eye gaze tracking score.

FIG. 7C is an explanatory diagram of an utterance tracking score.

FIG. 8 is an explanatory diagram illustrating an overview of a disease risk score computation model.

FIG. 9 is a flowchart illustrating an example of a processing procedure of generating a pareidolia test image.

FIG. 10 is a flowchart illustrating an example of a processing procedure of a pareidolia test.

FIG. 11 is a flowchart illustrating an example of a processing procedure of the pareidolia test.

FIG. 12A is a flowchart illustrating an example of a processing procedure of the pareidolia test.

FIG. 12B is a flowchart illustrating an example of a processing procedure of the pareidolia test.

FIG. 13A is a flowchart illustrating an example of a processing procedure of the pareidolia test.

FIG. 13B is a flowchart illustrating an example of a processing procedure of the pareidolia test.

FIG. 14A is an explanatory diagram illustrating a screen example of the user terminal.

FIG. 14B is an explanatory diagram illustrating a screen example of the user terminal.

FIG. 14C is an explanatory diagram illustrating a screen example of the user terminal.

FIG. 14D is an explanatory diagram illustrating a screen example of the user terminal.

FIG. 15A is an explanatory diagram illustrating a screen example of the user terminal.

FIG. 15B is an explanatory diagram illustrating a screen example of the user terminal.

FIG. 15C is an explanatory diagram illustrating a screen example of the user terminal.

FIG. 15D is an explanatory diagram illustrating a screen example of the user terminal.

FIG. 16A is an explanatory diagram of the pareidolia test of this embodiment.

FIG. 16B is an explanatory diagram of the pareidolia test of this embodiment.

FIG. 17 is a flowchart illustrating an example of a processing procedure of a pareidolia test of Embodiment 2.

FIG. 18 is a flowchart illustrating an example of a processing procedure of generating an evaluation table for the pareidolia test.

FIG. 19 is an explanatory diagram illustrating an example of the evaluation table for the pareidolia test.

FIG. 20 is an explanatory diagram illustrating an overview of a disease risk score computation model of Embodiment 3.

FIG. 21A is a flowchart illustrating an example of a processing procedure of a pareidolia test of Embodiment 3.

FIG. 21B is a flowchart illustrating an example of a processing procedure of the pareidolia test of Embodiment 3.

FIG. 22A is an explanatory diagram illustrating a screen example of the user terminal.

FIG. 22B is an explanatory diagram illustrating a screen example of the user terminal.

FIG. 22C is an explanatory diagram illustrating a screen example of the user terminal.

FIG. 22D is an explanatory diagram illustrating a screen example of the user terminal.

FIG. 22E is an explanatory diagram illustrating a screen example of the user terminal.

FIG. 22F is an explanatory diagram illustrating a screen example of the user terminal.

FIG. 22G is an explanatory diagram illustrating a screen example of the user terminal.

FIG. 22H is an explanatory diagram illustrating a screen example of the user terminal.

FIG. 22I is an explanatory diagram illustrating a screen example of the user terminal.

FIG. 22J is an explanatory diagram illustrating a screen example of the user terminal.

FIG. 23A is an explanatory diagram illustrating a verification result.

FIG. 23B is an explanatory diagram illustrating a verification result.

### DESCRIPTION

Hereinafter, a program, an information processing method, and an information processing device of the disclosure will be described with reference to the drawings illustrating embodiments thereof.

### Embodiment 1

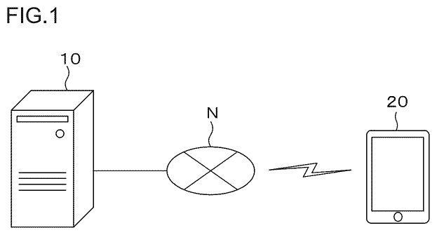

A description will be given of an information processing system that generates an image used in a pareidolia test (hereinafter referred to as a pareidolia test image) and performs a pareidolia test using the generated pareidolia test image. FIG. 1 is an explanatory diagram illustrating a configuration example of the information processing system. The information processing system of this embodiment includes a server 10 and a user terminal 20, and the server 10 and the user terminal 20 are connected for communication via a network N. The network N may be the Internet or a public communication line, or may be a LAN (Local Area Network) constructed in a facility where the information processing system is installed.

The server 10 is an information processing device capable of processing various types of information and transmitting and receiving information, and is, for example, a server computer, a personal computer, a workstation, etc. The server 10 is installed in a medical institution, a testing institution, a research institution, etc. The user terminal 20 is a terminal used by a subject who takes a pareidolia test using the server 10, and is a general-purpose information processing device such as a smartphone, a tablet terminal, or a personal computer. In addition, the user terminal 20 may be an HMD (Head Mounted Display) type information processing device worn and used by the subject, or may be a combination of a general-purpose information processing device and a display device such as an HMD. Since the subject takes the pareidolia test while holding the user terminal 20 in a hand, for example, the user terminal 20 is preferably a portable terminal. Note that the user of the user terminal 20 is not limited to the subject, but hereinafter, a person who uses the user terminal 20 will be collectively referred to as the subject. Furthermore, the user terminal 20 may be a terminal provided by a medical institution, etc. in addition to a terminal of the subject.

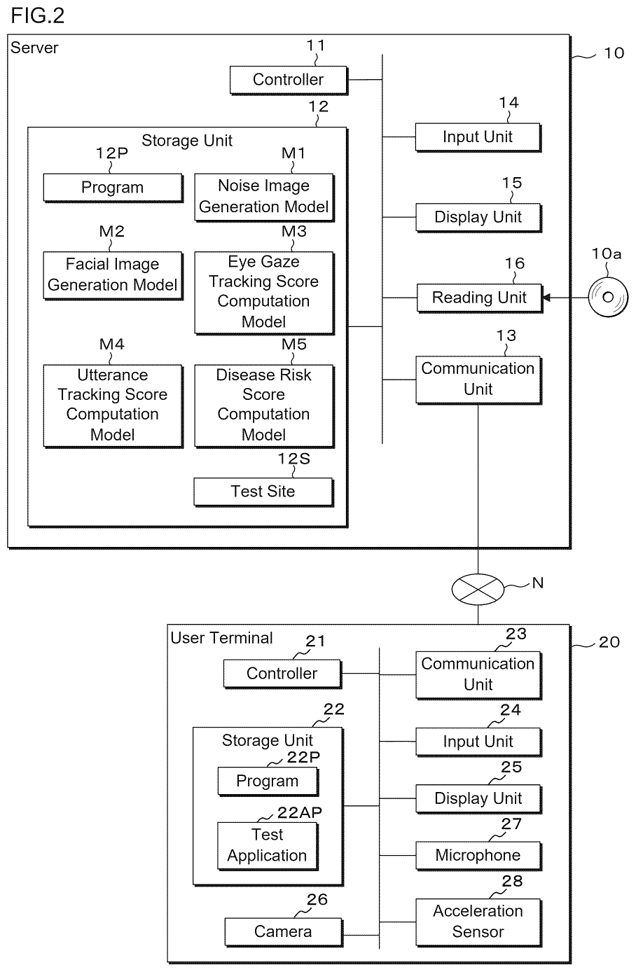

In the information processing system of this embodiment, the server 10 has a function as a web server, and provides a test site 12S (see FIG. 2) for conducting the pareidolia test via the network N. The server 10 transmits a screen for the pareidolia test to the user terminal 20 in response to a request from the user terminal 20, and the user terminal 20 displays a screen for the pareidolia test received from the server 10. The subject takes the pareidolia test in accordance with the screen for the pareidolia test displayed on the user terminal 20.

FIG. 2 is a block diagram illustrating a configuration example of the server 10 and the user terminal 20. The server 10 includes a controller 11, a storage unit 12, a communication unit 13, an input unit 14, a display unit 15, a reading unit 16, etc., and these units are connected via a bus. The controller 11 includes one or more processors such as a CPU (Central Processing Unit), an MPU (Micro-Processing Unit), a GPU (Graphics Processing Unit), and an AI chip (AI semiconductor). The controller 11 executes a program 12P stored in the storage unit 12 as appropriate to perform processing to be performed by the server 10.

The storage unit 12 includes a RAM (Random Access Memory), a flash memory, a hard disk, an SSD (Solid State Drive), etc. The storage unit 12 pre-stores the program 12P (program product) executed by the controller 11, various types of data required for executing the program 12P, etc. The program 12P is a basic program for controlling an operation of each unit of the server 10. In addition, the storage unit 12 temporarily stores data generated when the controller 11 executes the program 12P. In addition, the storage unit 12 stores a noise image generation model M1, a facial image generation model M2, an eye gaze tracking score computation model M3, an utterance tracking score computation model M4, and a disease risk score computation model M5, which will be described later. Each of the models M1 to M5 is assumed to be used as a program module included in artificial intelligence software. The storage unit 12 stores, as information defining each of the models M1 to M5, information on layers included in the respective models M1 to M5, information on nodes included in the respective layers, weights (coupling coefficients) between the nodes, etc. The storage unit 12 further stores the test site 12S. The test site 12S is a Web site for providing various types of information required for the pareidolia test to the terminal of the subject (user terminal 20). Each of the models M1 to M5 may be stored in another storage device connected to the server 10, or in another storage device with which the server 10 can communicate.

The communication unit 13 is a communication module for connection to the network N by wired or wireless communication, and transmits and receives information to and from other devices via the network N. The input unit 14 receives operation input by a user of the server 10, and sends a control signal corresponding to operation content to the controller 11. The display unit 15 is a liquid crystal display, an organic EL display, etc., and displays various types of information according to an instruction from the controller 11. A part of the input unit 14 and the display unit 15 may be an integrally configured touch panel. Note that the input unit 14 and the display unit 15 are not essential, and the server 10 may be configured to receive an operation through a connected computer, or to output information to be displayed to an external display device.

The reading unit 16 reads information stored in a portable storage medium 10a, such as a CD (Compact Disc), a DVD (Digital Versatile Disc), a USB (Universal Serial Bus) memory, an SD card, a micro SD card, a Compact Flash®, etc. The program 12P (program product) and various types of data stored in the storage unit 12 may be read by the controller 11 from the portable storage medium 10a via the reading unit 16 and stored in the storage unit 12, or may be downloaded by the controller 11 from another device via the communication unit 13 and stored in the storage unit 12.

The server 10 may be a multi-computer including a plurality of computers, or may be a virtual machine virtually constructed by software in one device. In addition, when the server 10 is configured as the server computer, the server 10 may be a local server installed in a medical institution, etc., or may be a cloud server connected for communication via a network such as the Internet. In the following description, the server 10 is assumed to be a single computer. In addition, the program 12P may be executed on a single computer, or may be executed on a plurality of computers connected to each other via the network N.

The user terminal 20 includes a controller 21, a storage unit 22, a communication unit 23, an input unit 24, a display unit 25, a camera 26, a microphone 27, an acceleration sensor 28, etc., and these units are connected via a bus. Each of the controller 21, the storage unit 22, the communication unit 23, the input unit 24, and the display unit 25 has the same configuration as that of each of the controller 11, the storage unit 12, the communication unit 13, the input unit 14, and the display unit 15 of the server 10, and thus a detailed description thereof will be omitted. Note that the storage unit 22 of the user terminal 20 stores a program 22P, which is a basic program for controlling an operation of each unit of the user terminal 20, as well as a test application program 22AP (hereinafter referred to as test application 22AP) for taking the pareidolia test provided by the server 10. The display unit 25 of the user terminal 20 preferably has a size allowing a pareidolia test image to be fully visually recognized, and preferably has a diagonal length of, for example, 6 inches or more.

The camera 26 is an imaging device having a lens and an imaging element, which performs imaging processing according to an instruction from the controller 21, for example, acquiring 30 or 15 pieces of image data (video) per second, and stores the acquired image data in the storage unit 22. The camera 26 is, for example, an in-camera provided in a smartphone, is provided at a position where a face of the subject visually recognizing the display unit 25 of the user terminal 20 can be captured, and captures eye movement of the subject. The camera 26 may be a visible camera capable of capturing visible light, or may be an infrared camera, and a plurality of cameras 26 may be provided. The microphone 27 collects sound and acquires voice data according to an instruction from the controller 21, and stores the acquired voice data in the storage unit 22. The microphone 27 is provided at a position where spoken voice of the subject operating the user terminal 20 can be captured, and captures the spoken voice of the subject. The camera 26 and the microphone 27 may be built into the user terminal 20, or may be configured to be externally attached to the user terminal 20. The acceleration sensor 28 detects a tilt angle of the user terminal 20 held by the subject, for example, a tilt angle of a display screen of the display unit 25. The user terminal 20 may have various sensors such as a gyro sensor in addition to the above-mentioned configuration.

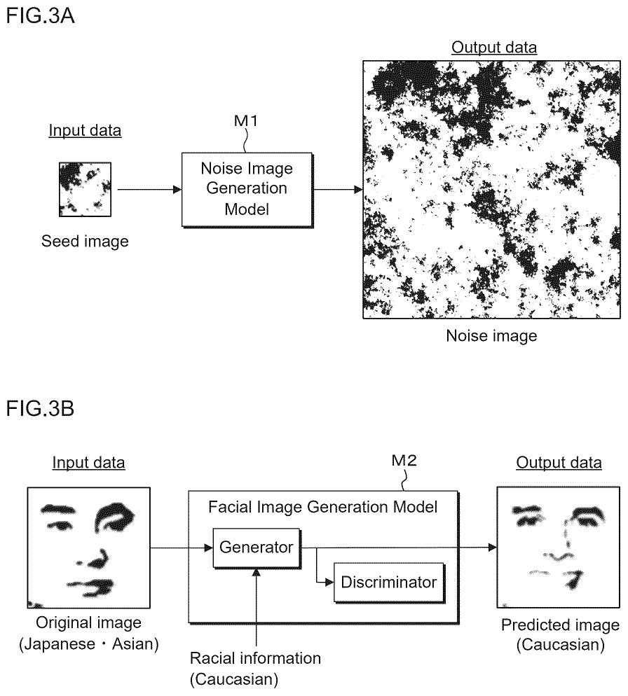

FIG. 3A is an explanatory diagram illustrating an overview of the noise image generation model M1. The noise image generation model M1 is configured, for example, using an algorithm of a Markov random field model. The noise image generation model M1 is configured to perform calculation to generate a large-sized noise image (texture) by performing texture synthesis using a small-sized seed image represented as a binary image when the seed image is input, and output the generated noise image (noise pattern image). The seed image is an image having at least one black pixel. The noise image preferably includes a noise pattern that can induce pareidolia, rather than a noise pattern that can be clearly determined to be noise (for example, a completely random noise pattern, a periodic noise pattern, or a white noise pattern). In order to generate such a noise image, the seed image is not an image of a white noise pattern, but an image generated assuming a noise pattern to be generated, and the seed image may be generated by an expert such as a doctor. In addition, the noise image generation model M1 is configured to be able to set a degree of randomness or periodicity of pixels (patches having a plurality of pixels) synthesized in texture synthesis by parameters in order to generate a noise pattern that can induce pareidolia. For example, by reducing randomness or increasing periodicity, it is possible to generate a noise pattern structured to some extent. Therefore, by appropriately setting the seed image and parameters, it is possible to realize the noise image generation model M1 that can generate a noise image including a noise pattern that can induce pareidolia. Note that the noise image generation model M1 is not limited to a configuration that generates a noise image by texture synthesis. In addition, the noise image generation model M1 is not limited to a configuration that uses the Markov random field model, and may be configured using other algorithms or a combination of a plurality of algorithms.

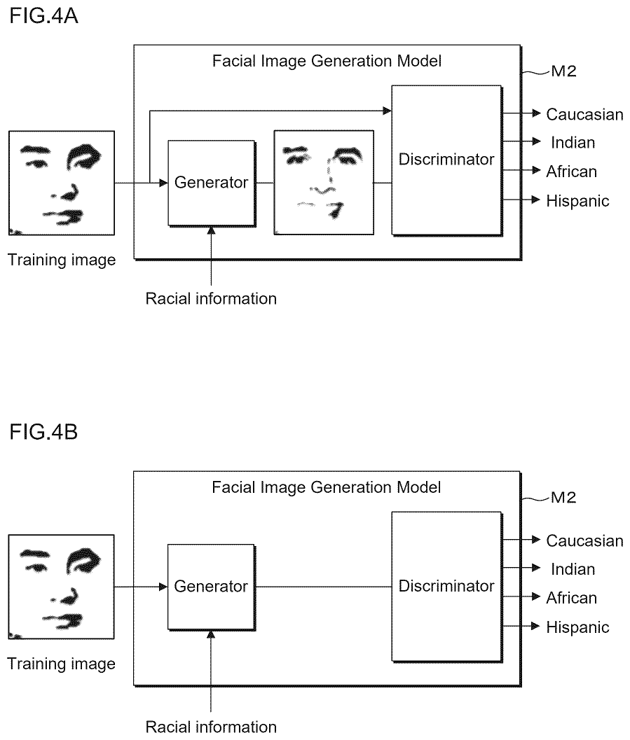

FIG. 3B to FIG. 4B are explanatory diagrams illustrating overviews of a facial image generation model M2. The facial image generation model M2 is a model trained to output a facial image (hereinafter referred to as a predicted image) of a person of a different race from that of a person in an input facial image (hereinafter referred to as an original image) represented as a binary image when the facial image is input. As illustrated in FIG. 3B to FIG. 4B, in the facial image generation model M2 of this embodiment, a binary facial image generated by extracting features of eyes, a nose, a mouth, and eyebrows from a photograph obtained by capturing a face of a person is used as the original image. FIG. 3B conceptually illustrates a state in which the facial image generation model M2 generates a predicted image from an original image, and FIG. 4A and FIG. 4B conceptually illustrate states when the facial image generation model M2 is trained. In addition, FIG. 4A illustrates a state when a discriminator is trained, and FIG. 4B illustrates a state when a generator is trained. FIG. 3B illustrates an example in which the facial image generation model M2 of this embodiment predicts a facial image (generates a predicted image) of a race other than Japanese (Asian) using an original image of a Japanese (Asian) as input. However, the disclosure is not limited to this configuration, and it is sufficient to adopt a configuration in which a predicted image of a second race different from a first race is generated from an original image of the first race.

The facial image generation model M2 is configured using, for example, a GAN (Generative Adversarial Network). The GAN includes a generator that generates output data from input data, and a discriminator that identifies a race of data (predicted image) generated by the generator, and the generator and the discriminator are trained in an adversarial manner through competition, thereby constructing a network. The generator is a module having an encoder that extracts latent variables from input data, and a decoder that generates output data from the extracted latent variables. The facial image generation model M2 of this embodiment is configured to allow the race of the predicted image to be set for the decoder possessed by the generator, and the generator is configured to output the predicted image of the race based on racial information set in the decoder. Races allowed to be set can be, for example, Caucasian, Indian, African, Hispanic, East Asian, Arab, etc. However, the disclosure is not limited thereto.

The facial image generation model M2 is generated by preparing training data in which an original image for training (hereinafter referred to as training image) is associated with racial information, and training a model using this training data. As the training image, it is possible to use a publicly available facial image of each race, for example, it is possible to use a binary facial image generated by extracting features of eyes, a nose, a mouth, and eyebrows from a photograph obtained by capturing a face of a person. The training image preferably includes facial images with different eye gaze directions. Note that, since the pareidolia test is a test designed for Japanese people, facial images in conventionally used pareidolia test images are Japanese (Asian) facial images in many cases. Therefore, Japanese (Asian) facial images collected from conventional pareidolia test images may be used as training images. The server 10 of this embodiment generates the facial image generation model M2 trained using training data to output a predicted image of a different race from that of an input original image.

In a training process, the server 10 alternately updates parameters of the discriminator (such as weights between neurons) illustrated in FIG. 4A and parameters of the generator illustrated in FIG. 4B, and ends training when change in an error function converges. In updating the parameters of the discriminator, the server 10 fixes the parameters of the generator and then inputs a training image and racial information to the generator. Note that, in addition to being input to the generator, the racial information may be preset in the decoder of the generator. The generator receives input of training images and generates a predicted image as output data based on the racial information set in the decoder. Then, the server 10 inputs a pair of a training image and a predicted image, which correspond to input and output of the generator, to the discriminator, and causes the discriminator to identify a race of the predicted image. The discriminator receives input of the training image and the predicted image (the image generated by the generator), performs calculation to identify the race of the predicted image, and outputs a calculation result. Note that the discriminator has output nodes to which preset races are associated, respectively, and outputs a probability (certainty) of identifying each race from each output node. An output value from each output node is, for example, a value between 0 and 1, and the sum of probabilities output from the respective output nodes is 1.0(100%). The server 10 uses racial information for training as a ground truth label to train the discriminator so that an output value from an output node corresponding to a race of the ground truth label approaches 1 and output values from other output nodes approach 0. Specifically, the server 10 compares an output value from each output node of the discriminator with a value corresponding to the ground truth label (specifically, 1 for the output node corresponding to the ground truth label and 0 for the other output nodes), and updates the parameters of the discriminator so that the two values are close to each other. The updated parameters are weights (coupling coefficients) between nodes, etc. in the discriminator, and the backpropagation method, the steepest descent method, etc. can be used as a parameter optimization method.

In updating the parameters of the generator, the parameters of the discriminator are fixed and then training is performed as illustrated in FIG. 4B. Here, the server 10 inputs training images and racial information to the generator, and updates the parameters of the generator so that, when a predicted image generated by the generator is input to the discriminator, a predicted image identified as racial information for training by the discriminator is generated. Here, the updated parameters are weights (coupling coefficients) between nodes, etc. in the generator, and the backpropagation method, the steepest descent method, etc. can be used as a parameter optimization method. In this way, as illustrated in FIG. 3B, the facial image generation model M2, which outputs a predicted image of a race designated by racial information when an original image is input, is generated. Note that, when a facial image is actually predicted from an original image using the facial image generation model M2, the server 10 uses only the generator as illustrated in FIG. 3B.

The facial image generation model M2 may be configured using DCGAN (Deep Convolutional GAN), TP-GAN (Two Pathway-GAN), SRGAN (Super Resolution GAN), CycleGAN, StarGAN, etc. When using TP-GAN, it is possible to realize the facial image generation model M2 that outputs a predicted image whose eye gaze direction is different from that of an original image. In addition, the facial image generation model M2 is not limited to GAN, and may be a model based on VAE (Variational Autoencoder), a neural network such as CNN (Convolutional Neural Network) (for example, U-net), or other learning algorithms, or may be configured by combining a plurality of learning algorithms. In addition, the facial image generation model M2 is not configured to receive input of racial information, and may be configured to receive only input of an original image and output predicted images of a plurality of races set in advance. In this case, an image used in the pareidolia test may be selected from the predicted images (facial images) of the plurality of races output from the facial image generation model M2. In addition, the facial image generation model M2 may be generated and prepared for each race of the predicted images by previously setting information of each race in the decoder of the generator. Furthermore, the original image input to the facial image generation model M2 is not limited to a human facial image, and the facial image generation model M2 may be configured to predict a human facial image of each race from, for example, an animal facial image.

The server 10 prepares the noise image generation model M1 and the facial image generation model M2 as described above in advance and uses the models when generating a pareidolia test image. Specifically, the server 10 uses the noise image generation model M1 to generate a noise image from a seed image, and uses the facial image generation model M2 to generate a facial image (predicted image) of another race from a facial image (original image) of a Japanese (Asian). The server 10 then synthesizes the generated noise image and the generated facial image (predicted image) to generate a pareidolia test image. Note that a predicted image is preferably generated based on facial images with a plurality of eye gaze directions (for example, a frontward direction, a leftward direction, a rightward direction, an upward direction, and a downward direction) as the original image. In this case, it is possible to generate a pareidolia test image having a facial image with any eye gaze direction.

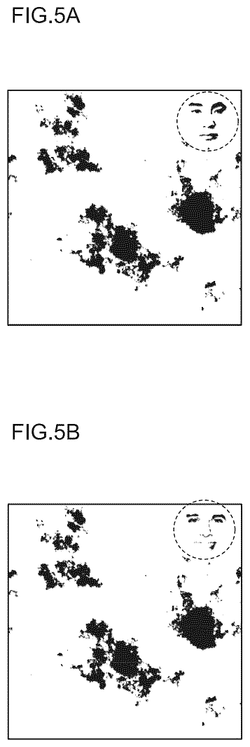

FIG. 5A and FIG. 5B are explanatory diagrams illustrating examples of pareidolia test images. FIG. 5A and FIG. 5B illustrate pareidolia test images obtained by synthesizing different facial images with respect to the same noise image. In each of the examples illustrated in FIG. 5A and FIG. 5B, a facial image is synthesized in a top right region (region surrounded by a dashed line in the figure) of the pareidolia test image. By such processing, in this embodiment, for example, it is possible to generate a facial image according to a race of a subject, and to generate a pareidolia test image according to the subject using the generated facial image. Note that a region where a facial image is synthesized with a noise image may be any region in a randomly selected quadrant, any region in a quadrant selected in a predetermined order, or a region designated by the user. In addition, depending on the density of black pixels in the noise image, a region where the proportion of black pixels is less than a predetermined value may be set as a synthesis region for the facial image, and when the noise image is generated, pixels in a region where the facial image is synthesized may be set as white pixels. In this way, it is possible to synthesize a facial image in a region having few black pixels in the noise image, and to generate a pareidolia test image allowing a facial region to be appropriately recognized.

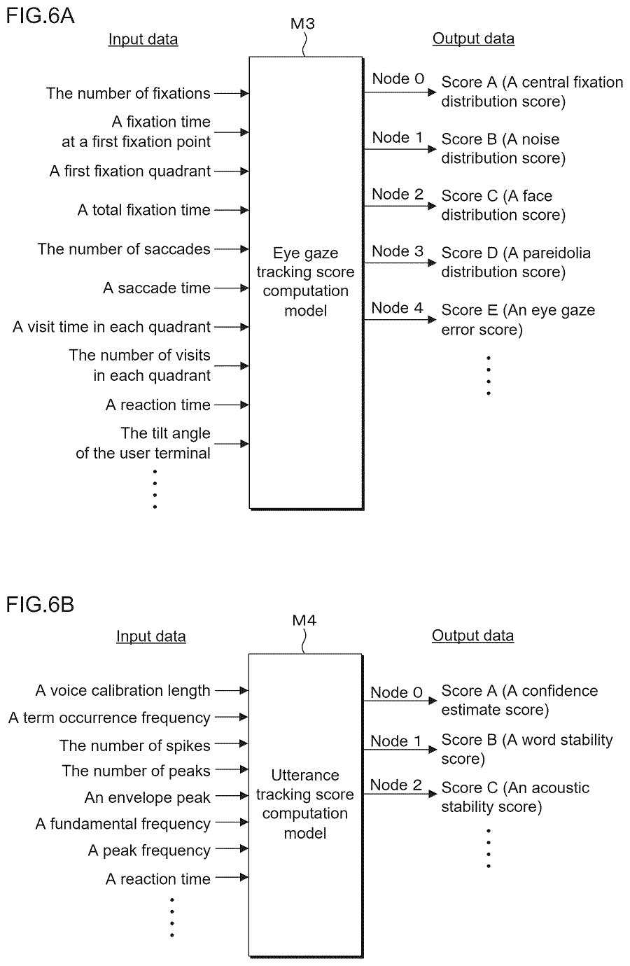

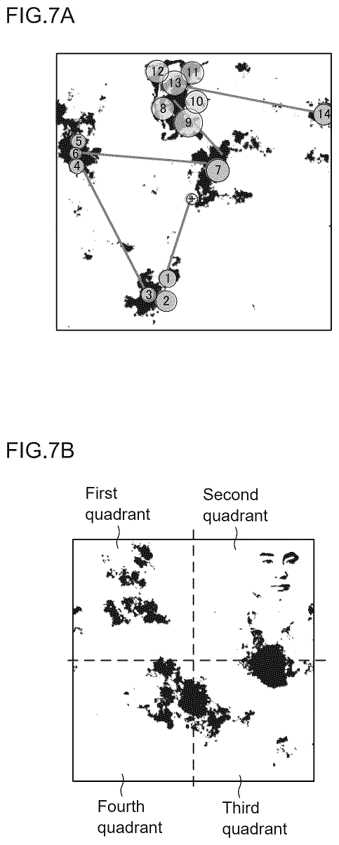

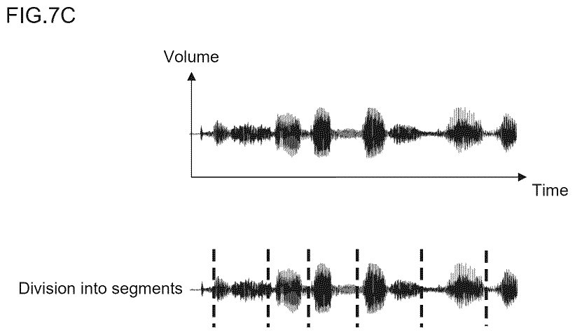

FIG. 6A is an explanatory diagram illustrating an overview of the eye gaze tracking score computation model M3, FIG. 6B is an explanatory diagram illustrating an overview of the utterance tracking score computation model M4, FIG. 7A and FIG. 7B are explanatory diagrams of eye gaze tracking scores, and FIG. 7C is an explanatory diagram of an utterance tracking score. The eye gaze tracking score computation model M3 is trained to receive input of a tracking result obtained by tracking an eye gaze (eye movement) of a subject taking a pareidolia test, perform calculation to predict a score for eye movement of the subject (hereinafter referred to as an eye gaze tracking score) based on each piece of input information, and output a calculation result. The eye gaze tracking score computation model M3 is configured using algorithms such as Support Vector Machine (SVM), Random Forest, Convolutional Neural Network (CNN), Recurrent Neural Network (RNN), Long Short-Term Memory (LSTM), and Transformer. Note that the eye gaze tracking score computation model M3 may be configured using other learning algorithms or may be configured by combining a plurality of learning algorithms.

The eye gaze tracking score computation model M3 has an input layer, an intermediate layer, and an output layer. The input layer has a plurality of input nodes, and each input node is associated with information to be input. The information input to the eye gaze tracking score computation model M3 is a result of tracking the eye gaze (eye movement) when the subject visually recognizes a pareidolia test image, and includes the number of fixations for the pareidolia test image, a fixation time at a first fixation point, a first fixation quadrant, a total fixation time, the number of saccades, a saccade time, a visit time and the number of visits in each quadrant, a reaction time, and the tilt angle of the user terminal 20 during the pareidolia test. Note that, referring to the quadrants, as illustrated in FIG. 7B, respective regions obtained by dividing the pareidolia test image into four equal parts by a vertical center line and a horizontal center line are set to first to fourth quadrants starting from a top left region in a clockwise direction. In this embodiment, a process of tracking the eye gaze is performed based on the four quadrants. However, the tracking process may be performed for each region obtained by dividing the pareidolia test image into parts, the number of which is other than four. For example, the pareidolia test image may be divided into nine regions, 3 vertical×3 horizontal, and the tracking process may be performed for each region. The number of fixations indicates the number of fixation points, where a spot viewed (steadily gazed) by the subject for a predetermined time (for example, 0.1 seconds) or more for one pareidolia test image is defined as a fixation point. The fixation time at the first fixation point indicates a time spent continuously viewing the first fixation point for one pareidolia test image, and the first fixation quadrant indicates a quadrant having the first fixation point. The total fixation time indicates the sum of fixation times at respective fixation points for one pareidolia test image. The number of saccades indicates the number of times of occurrence of eye movement referred to as a saccade generated when changing the fixation point for one pareidolia test image, and the saccade time indicates the total duration of each saccade. The visit time indicates a time during which an eye gaze is directed at each quadrant of one pareidolia test image, and adopts the sum of the fixation time and the saccade time in each quadrant. The number of visits indicates the number of fixations and saccades in each quadrant of one pareidolia test image, and adopts the sum of the number of fixations and the number of saccades in each quadrant. The reaction time indicates a time from display of one pareidolia test image to reaction of the subject. Reaction of the subject means performance of an operation on a region appearing on a face in the image, an operation on a “next” button, or utterance of “yes” or “no”. The tilt angle of the user terminal 20 is an angle detected by the acceleration sensor 28, and indicates, for example, the tilt angle of the display screen. Each of these pieces of information is input to the eye gaze tracking score computation model M3 via the corresponding input node. Each piece of the information input to the eye gaze tracking score computation model M3 is not limited to the above-mentioned information, and other parameters related to eye movement that can be used to predict symptoms of neuropsychiatric disorders may be used. For example, in each pareidolia test image, a quadrant having a facial image may be defined as a face area, and a quadrant not having a facial image may be defined as a noise area. Information indicating whether the first to fourth quadrants in each pareidolia test image are face areas or noise areas may be input to the eye gaze tracking score computation model M3.

As these pieces of information, each piece of information for all pareidolia test images used in the pareidolia test may be input, each piece of information for some of the pareidolia test images may be input, or an average value of each piece of information for each pareidolia test image may be input. In addition, the reaction time may include any one or more of a reaction time of an image responding to a pareidolia test image including a facial image with “yes” or an average value thereof, a reaction time of an image responding to a pareidolia test image including only a noise image with “no” or an average value thereof, a reaction time of an image responding to a pareidolia test image including only a noise image with “yes” or an average value thereof, and a reaction time of an image responding to a pareidolia test image including a facial image with “no” or an average value thereof. These pieces of information can be acquired by the process of tracking the eye gaze, and are obtained from an eye gaze map indicating a result of tracking the eye gaze. FIG. 7A illustrates an example of the eye gaze map. The eye gaze map illustrated in FIG. 7A indicates a trajectory of the eye gaze of the subject, each fixation point is indicated by a circuit, and a movement trajectory (saccade) between fixation points is indicated by as a straight line. An order of fixation is associated with a circuit indicating each fixation point, and a longer fixation time is indicated by a larger circuit.

The intermediate layer calculates an output value from each piece of information input through the input layer using various functions, thresholds, etc., and outputs the calculated output value to the output layer. The output layer has a plurality of output nodes, an item indicating an eye gaze tracking score for eye movement of the subject is associated with each output node, and the score of the associated item is output from each output node. In the example illustrated in FIG. 6A, output node 0 outputs, as score A, a central fixation distribution score representing how eye movement returns to a central or general fixed position in a neutral state. Output node 1 outputs, as score B, a noise distribution score representing how the noise area in the pareidolia test image was visually recognized, and output node 2 outputs, as score C, a face distribution score representing how the face area in the pareidolia test image was visually recognized. Output node 3 outputs, as score D, a pareidolia distribution score representing how the entire pareidolia test image was visually recognized. Output node 4 outputs, as score E, an eye gaze error score representing a degree or percentage to or at which the eye gaze is out of the pareidolia test image and a result of tracking the eye gaze cannot be used for the pareidolia test. Note that the item associated with each output node is not limited to the example illustrated in FIG. 6A, and it is possible to use an item allowing prediction of symptoms of neuropsychiatric disorders, an item affected by neuropsychiatric disorders, an item that can be used to classify neuropsychiatric disorders, etc. With the above-mentioned configuration, the eye gaze tracking score computation model M3 outputs each score for eye movement of the subject when information related to the result of tracking eye gaze of the subject is input.

The eye gaze tracking score computation model M3 is generated by machine-training a learning model using training data including each piece of information on the result of tracking eye gaze for training and the score of each item of the eye gaze tracking score corresponding to the result of tracking eye gaze. For a healthy person (a person not having a neuropsychiatric disorder), the training data is generated by assigning a score (ground truth) of each item obtained from eye movement of the healthy person to a result of tracking eye gaze obtained when the person takes a pareidolia test. In addition, for patients having neuropsychiatric disorders such as Lewy body dementia, Alzheimer's disease, Parkinson's disease, or schizophrenia, the training data is generated by assigning a score (ground truth) of each item obtained from eye movement of each patient having a neuropsychiatric disorder to a result of tracking eye gaze obtained when the patient takes a pareidolia test. Ground truth scores of each item for eye movement of the healthy person and the patient having the neuropsychiatric disorder may be values determined by an expert such as a doctor. The training data generated in this manner is stored, for example, in a training DB (not illustrated) prepared in the storage unit 12, and is used during a training process.

The eye gaze tracking score computation model M3 is trained so that an output value from each output node approaches a ground truth score of each item when each piece of information on a result of tracking eye gaze included in training data is input. In a training process, the eye gaze tracking score computation model M3 performs calculation based on each piece of information on the input result of tracking eye gaze, and calculates an output value from each output node. Then, the eye gaze tracking score computation model M3 compares the calculated output value of each output node with the ground truth score of each item, and optimizes parameters used in a calculation process so that the two values are close to each other. The parameters are weights between neurons in the eye gaze tracking score computation model M3, etc. A method of optimizing the parameters is not particularly limited, but the backpropagation method, the steepest descent method, etc. can be used. In this way, it is possible to obtain the eye gaze tracking score computation model M3 trained to predict an eye gaze tracking score for eye movement of the subject and output a predicted score for each item when each piece of information on the result of tracking eye gaze of the subject is input.

The eye gaze tracking score computation model M3 is not limited to the configuration illustrated in FIG. 6A. For example, each piece of information input to the eye gaze tracking score computation model M3 may include, in addition to the above-mentioned information, any one or more of age, sex, medical history, treatment history, medication history, etc. of the subject. In this case, the eye gaze tracking score computation model M3 is configured to predict an eye gaze tracking score for determining a possibility of neuropsychiatric disorders based on not only a result of tracking eye gaze but also various types of information of the patient. In addition, the eye gaze tracking score computation model M3 may be configured to receive input of an NPT score (score of the pareidolia test) and a health profile score (or an answer to a questionnaire for a health profile) described later in addition to the above-mentioned information. Furthermore, for subjects diagnosed with pareidolia or neuropsychiatric disorders, the eye gaze tracking score computation model M3 may be configured to receive a period of time since diagnosis. Note that, in the above-mentioned example, a quadrant having a facial image is defined as a face area, and other quadrants are defined as noise areas. However, more precisely, only a facial image part may be defined as a face area, the other parts may be defined as noise areas, information indicating whether each fixation point corresponds to a face area or a noise area may be acquired, and the information may be included in the input of the eye gaze tracking score computation model M3.

The utterance tracking score computation model M4 illustrated in FIG. 6B is trained to receive input of a tracking result of tracking spoken voice of the subject taking a pareidolia test, perform calculation to predict a score for the spoken voice of the subject (hereinafter referred to as utterance tracking score) based on each piece of input information, and output a calculation result. The utterance tracking score computation model M4 has a configuration similar to that of the eye gaze tracking score computation model M3, and is configured using algorithms such as SVM, random forest, CNN, RNN, LSTM, and Transformer. The utterance tracking score computation model M4 may be configured using other learning algorithms, or may be configured by combining a plurality of learning algorithms.

Information input to an input layer of the utterance tracking score computation model M4 is information related to spoken voice uttered by the subject during a pareidolia test, and includes a voice calibration length, a term occurrence frequency, the number of spikes, the number of peaks, an envelope peak, a fundamental frequency, a peak frequency, and a reaction time. The voice calibration length indicates a time required for the subject to utter a given sentence in calibration in an utterance tracking process performed before the start of the pareidolia test. The term occurrence frequency indicates a ratio of the number of times of occurrence of “yes” and “no” to the total number of utterance words during the pareidolia test (specifically, a value obtained by dividing the number of times of occurrence of “yes” and “no” by the number of times of occurrence of all words). The term occurrence frequency can be measured, for example, by the TF-IDF (Term Frequency-Inverse Document Frequency) algorithm. The number of spikes indicates the number of times of occurrence of a spike, which is an instantaneous increase in volume. Occurrence of a spike can be determined based on whether or not the volume has sharply increased by a predetermined amount or more within a predetermined time, and a criterion for this determination can be arbitrarily set. When volume of a predetermined value or more occurs and then the volume drops, a maximum volume value at this time is set to a peak, and the number of peaks indicates the number of times of occurrence of the peak. The envelope peak indicates an average movement line of each peak occurring in spoken voice, the fundamental frequency indicates a lowest frequency among frequencies included in the spoken voice, and the peak frequency indicates a frequency having a largest amplitude value among the frequencies included in the spoken voice. The reaction time indicates a time from display of the pareidolia test image to reaction of the subject. Each of these pieces of information is input to the utterance tracking score computation model M4 via the corresponding input node. Each piece of information input to the utterance tracking score computation model M4 is not limited to the above-mentioned information, and other parameters related to spoken voice that can be used to predict symptoms of neuropsychiatric disorders may be used. In addition, in the utterance tracking process, when content of utterance of the subject is detected (recognized), text data of the detected utterance content may be input to the utterance tracking score computation model M4.

With regard to these pieces of information, for all pareidolia test images used in the pareidolia test, each piece of information collected during display of the images may be input, each piece of information collected for some pareidolia test images may be input, or an average value of respective pieces of information collected during displaying the respective images may be input. In addition, the reaction time may include any one or more of a reaction time of an image responding to a pareidolia test image including a facial image with “yes” or an average value thereof, a reaction time of an image responding to a pareidolia test image including only a noise image with “no” or an average value thereof, a reaction time of an image responding to a pareidolia test image including only a noise image with “yes” or an average value thereof, and a reaction time of an image responding to a pareidolia test image including a facial image with “no” or an average value thereof. These pieces of information can be acquired by a process of tracking the spoken voice, and are obtained from voice feature information that indicates a result of tracking the spoken voice. FIG. 7C illustrates an example of a voice signal of the spoken voice, and represents the spoken voice of the subject as a waveform graph with a horizontal axis representing time and a vertical axis representing volume. Spoken voice corresponding to one pareidolia test image is acquired each time image display is switched. For example, division into segments is performed for each word uttered by the subject, voice feature information is acquired for each segment, and each feature corresponding to one test image is acquired.

Each item indicating an utterance tracking score for the spoken voice of the subject is associated with each output node of the output layer of the utterance tracking score computation model M4, and the score of the associated item is output from each output node. In the example illustrated in FIG. 6B, output node 0 outputs, as score A, a confidence estimate score for metacognitive ability to estimate a degree of pareidolia. Metacognitive ability is the ability to objectively recognize and understand what one is aware of, and is the ability affected by pareidolia (a visual hallucination symptom). Output node 1 outputs, as score B, a word stability score, indicating a ratio of the number of times of “yes” and “no” which are answers in the pareidolia test to the number of uttered words. Output node 2 outputs, as score C, an acoustic stability score indicating a degree to which acoustics in the spoken voice are stable. Stable acoustics are, for example, sound having nearly constant volume, sound having nearly constant pitch, etc. Note that the item associated with each output node is not limited to the example illustrated in FIG. 6B, and it is possible to use an item allowing prediction of symptoms of neuropsychiatric disorders, an item affected by neuropsychiatric disorders, an item that can be used to classify neuropsychiatric disorders, etc. With the above-mentioned configuration, the utterance tracking score computation model M4 outputs each score for spoken voice of the subject when information related to the result of tracking spoken voice of the subject is input.

Note that metacognitive ability has traditionally been studied in terms of a level of visual recognition using a metacognitive procedure. Typically, when cognitive ability is deficient (for example, in the case of a patient having a low cognitive state, such as dementia), subjective evaluation tends to be low or inappropriate. In general, metacognitive sensitivity is evaluated by measuring how confident the subject is in distinguishing between correct determination (when determining that an image including a face has a face) and incorrect determination (determining that an image not including a face has a face). For example, in a typical visual-only test such as the pareidolia test, a level of metacognitive functioning is evaluated by the subject responding to the pareidolia test image with presence or absence of a face and then, for example, answering a question “How confident are you in your answer?”. Answers to this question are made, for example, on a Likert scale from 1 (not at all confident) to 5 (very confident), and a level of metacognitive ability is calculated based on the presence or absence of confidence of the subject for each pareidolia test image. Meanwhile, in this embodiment, by using the utterance tracking score computation model M4, a reliability score for metacognitive ability (level of metacognitive ability) is calculated based on features of voice (volume (peak), intonation, reaction time, etc.) uttered by the subject during the pareidolia test. In this way, by calculating the level of metacognitive ability of the subject from a result of tracking spoken voice of the subject, the subject can obtain a level of metacognitive ability that clearly reflects a psychological process of the subject without the subject being conscious of presence or absence of confidence.

The utterance tracking score computation model M4 is generated by machine-training a learning model using training data including each piece of information on a result of tracking utterance for training and a score for each item of an utterance tracking score corresponding to the utterance tracking result. For a healthy person (person not having a neuropsychiatric disorder), the training data is generated by assigning a score (ground truth) of each item obtained from spoken voice of the healthy person to an utterance tracking result acquired when the person takes the pareidolia test. In addition, for a patient having a neuropsychiatric disorder such as Lewy body dementia, Alzheimer's disease, Parkinson's disease, or schizophrenia, the training data is generated by assigning a score (ground truth) of each item obtained from spoken voice of a patient having each neuropsychiatric disorder to an utterance tracking result acquired when the patient takes the pareidolia test. Ground truth scores of each item for spoken voice of the healthy person and the patient having the neuropsychiatric disorder may be values determined by an expert such as a doctor. The training data generated in this manner is stored, for example, in the training DB (not illustrated) prepared in the storage unit 12, and is used during a training process.

The utterance tracking score computation model M4 is trained so that an output value from each output node approaches a ground truth score of each item when each piece of information on the utterance tracking result included in the training data is input. In the training process, the utterance tracking score computation model M4 performs calculation based on each piece of input information on the utterance tracking result, and calculates an output value from each output node. Then, the utterance tracking score computation model M4 compares the calculated output value of each output node with the ground truth score of each item, and optimizes parameters used in the calculation process so that the two values are close to each other. Here, parameters such as weights between neurons in the utterance tracking score computation model M4 are optimized using the backpropagation method, the steepest descent method, etc. In this way, it is possible to obtain the utterance tracking score computation model M4 trained to predict an utterance tracking score for spoken voice of the subject and output a predicted score for each item when each piece of information on the result of tracking utterance of the subject is input.

The utterance tracking score computation model M4 is not limited to the configuration illustrated in FIG. 6B. For example, each piece of information input to the utterance tracking score computation model M4 may include any one or more of age, sex, medical history, treatment history, medication history, etc. of the subject in addition to the above-mentioned information. In this case, the utterance tracking score computation model M4 is configured to predict an utterance tracking score for determining a possibility of a neuropsychiatric disorder based on not only a result of tracking spoken voice but also various types of information of the patient. In addition, the utterance tracking score computation model M4 may be configured to receive input of the tilt angle of the user terminal 20 during the pareidolia test. In addition, the utterance tracking score computation model M4 may be configured to receive input of an NPT score (score of the pareidolia test) and a health profile score (or an answer to a questionnaire for the health profile), which will be described later, in addition to the above-mentioned information. Furthermore, the utterance tracking score computation model M4 may be configured to receive input of an elapsed period from diagnosis for a subject diagnosed with pareidolia or a neuropsychiatric disorder.

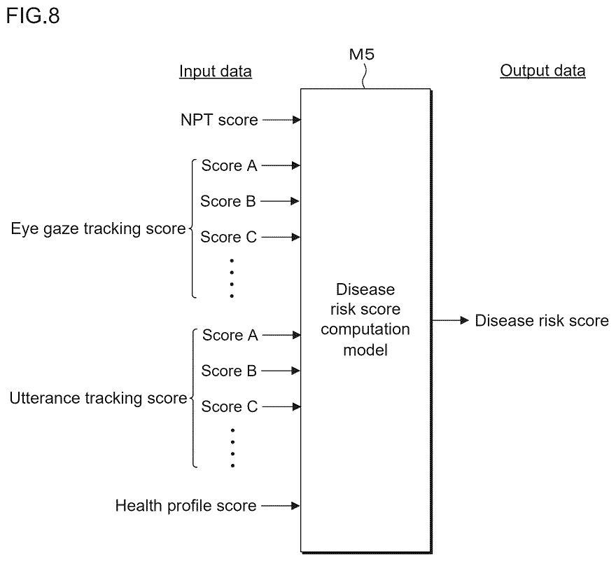

FIG. 8 is an explanatory diagram illustrating an overview of the disease risk score computation model M5. The disease risk score computation model M5 illustrated in FIG. 8 is trained to receive input of each score collected in each test performed in the pareidolia test in this embodiment, perform calculation to predict a score indicating a possibility that the subject has a neuropsychiatric disorder (hereinafter referred to as a disease risk score) based on each input score, and output a calculation result. The disease risk score computation model M5 is configured using algorithms such as SVM, random forest, CNN, RNN, LSTM, and Transformer. The disease risk score computation model M5 may be configured using other learning algorithms, or may be configured by combining a plurality of learning algorithms.

Information input to an input layer of the disease risk score computation model M5 includes an NPT (Noise Pareidolia Test) score which is a result of the pareidolia test, an eye gaze tracking score based on a result of tracking eye gaze during the pareidolia test, an utterance tracking score based on an utterance tracking result, and a health profile score based on an answer to a questionnaire for a health profile conducted together with the pareidolia test. The eye gaze tracking score includes each score specified using the above-mentioned eye gaze tracking score computation model M3, and the utterance tracking score includes each score specified using the above-mentioned utterance tracking score computation model M4. A process of calculating the NPT score and the health profile score will be described later. Each of these scores is input to the disease risk score computation model M5 via the corresponding input node. Each score input to the disease risk score computation model M5 is not limited to the above-mentioned scores, and other parameters that can be used to predict symptoms of neuropsychiatric disorders may be used.

An output layer of the disease risk score computation model M5 has one output node, and outputs a disease risk score (a risk score related to a neuropsychiatric disorder) indicating a possibility that the subject has a neuropsychiatric disorder. With this configuration, the disease risk score computation model M5 outputs a disease risk score for the subject when each score acquired when the subject took the pareidolia test is input. Note that the disease risk score computation model M5 is configured to output a disease risk score normalized to a value between 0 and 10, with 0 indicating a lowest risk of neuropsychiatric disorders and 10 indicating a highest risk of neuropsychiatric disorders. In addition, the disease risk score computation model M5 is configured to weight each input score according to importance thereof, and is configured to, for example, weight the NPT score heavily since the NPT score is more important than the health profile score. In addition, in addition to a configuration having one output node for calculating a disease risk score, the disease risk score computation model M5 may have a configuration having a plurality of output nodes with which a plurality of disease risk scores is associated, respectively, to output certainty for the associated disease risk score from each output node.

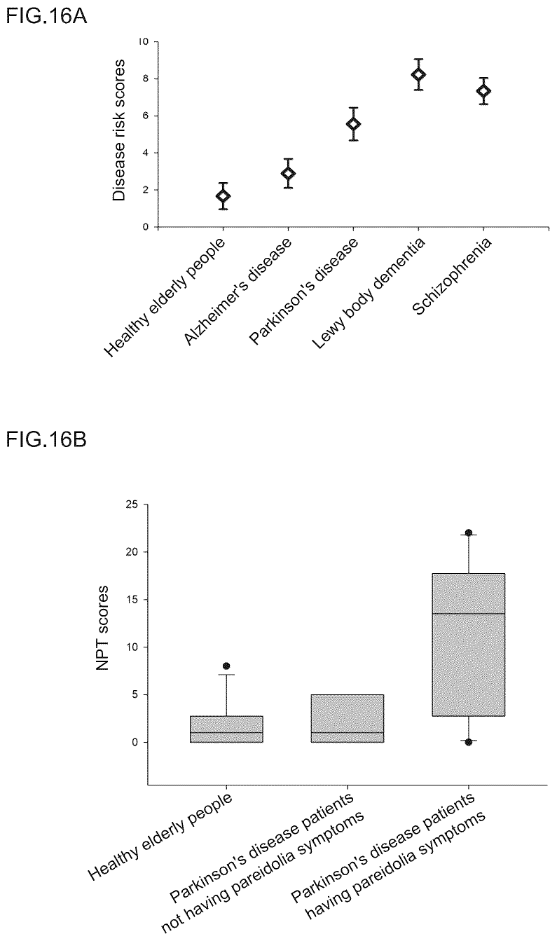

The disease risk score computation model M5 is generated by machine-training a learning model using training data including each score for training and a disease risk score indicating a possibility that a patient having the corresponding score has a neuropsychiatric disorder. For a healthy person (a person not having a neuropsychiatric disorder), the training data is generated by assigning a disease risk score (ground truth) indicating that the person is healthy to each score acquired when the person takes the pareidolia test. In addition, for a patient having a neuropsychiatric disorder such as Lewy body dementia, Alzheimer's disease, Parkinson's disease, or schizophrenia, the training data is generated by assigning a disease risk score (ground truth) indicating that the patient has each neuropsychiatric disorder to each score acquired when the patient takes the pareidolia test. The disease risk scores (ground truth scores) indicating that the person is healthy and the patient has the neuropsychiatric disorder can be values determined by an expert such as a doctor. As illustrated in FIG. 16A, since the disease risk scores that can be taken by the healthy person and the patient having the neuropsychiatric disorder are in different ranges, for example, scores increasing in order of healthy person, Alzheimer's disease, Parkinson's disease, schizophrenia, and Lewy body dementia may be used as disease risk scores (ground truth scores) for the healthy person and each neuropsychiatric disorder. For example, the training data generated in this manner is stored in the training DB prepared in the storage unit 12, and used during a training process.

The disease risk score computation model M5 is trained so that an output value from an output node approaches the disease risk score of the ground truth when each score included in the training data is input. In the training process, the disease risk score computation model M5 performs calculation based on each input score and computes the output value from the output node. The disease risk score computation model M5 then compares the computed output value of the output node with the disease risk score of the ground truth, and optimizes parameters used in a calculation process so that the two values are close to each other. Here, parameters such as weights between neurons in the disease risk score computation model M5 are optimized using the backpropagation method, the steepest descent method, etc. In this way, it is possible to obtain the disease risk score computation model M5 trained to predict and output a disease risk score for a subject when each score acquired when the subject takes the pareidolia test is input.

The disease risk score computation model M5 is not limited to the configuration illustrated in FIG. 8. For example, as each piece of information input to the disease risk score computation model M5, any one or more of age, sex, medical history, treatment history, medication history, etc. of the subject may be input in addition to each of the above-mentioned scores. In this case, the disease risk score computation model M5 is configured to predict a disease risk score indicating a possibility that the subject has a neuropsychiatric disorder based on not only each of the scores in the pareidolia test but also various types of information of the patient. In addition, the disease risk score computation model M5 is not limited to a configuration in which all of NPT score, eye gaze tracking score, utterance tracking score, and health profile score are input, and may have a configuration in which some of these scores are input. For example, in addition to the NPT score, any one or two of the eye gaze tracking score, utterance tracking score, and health profile score may be input.

Each of the models M1 to M5 may be trained by the server 10 or by another training device. For example, each of the trained models M1 to M5 generated by being trained using the other training device is downloaded from the training device to the server 10 via the network N or the portable storage medium 10a, and stored in the storage unit 12. Note that, for the trained facial image generation model M2, only the generator that generates a predicted image from an original image may be downloaded from the training device to the server 10.

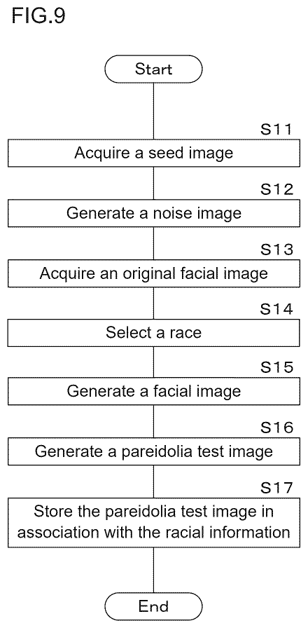

Hereinafter, a description will be given of processing performed by each device in the information processing system of this embodiment. First, a description will be given of processing in which the server 10 generates a pareidolia test image using the noise image generation model M1 and the facial image generation model M2. FIG. 9 is a flowchart illustrating an example of a processing procedure of generating a pareidolia test image. The controller 11 of the server 10 executes the following processing according to the program 12P stored in the storage unit 12. The controller 11 of the server 10 executes the following processing at any timing or at the timing when the subject takes the pareidolia test. In addition, it is assumed that a seed image and an original facial image are prepared in advance and stored in the storage unit 12.

The controller 11 of the server 10 acquires a seed image used to generate a noise image (S11). The controller 11 may read the seed image from the storage unit 12, may acquire the seed image from another information processing device via the communication unit 13, or may read the seed image from the portable storage medium 10a by the reading unit 16. The controller 11 generates a noise image based on the acquired seed image (S12). Specifically, the controller 11 inputs a seed image to the noise image generation model M1 and acquires a noise image output from the noise image generation model M1. Note that, when the noise image generation model M1 has configurable parameters, the controller 11 may set a set value input via the input unit 14 or a set value registered in advance for each parameter to generate a noise image.

Next, the controller 11 acquires an original facial image (original image) for generating a facial image to be used in the pareidolia test image (S13). The controller 11 may read the original image from the storage unit 12, may acquire the original image from another information processing device via the communication unit 13, or may read the original image from the portable storage medium 10a by the reading unit 16. The controller 11 selects a race of the facial image to be generated (S14). For example, the controller 11 selects any one of selectable races prepared in advance, such as Caucasian, Indian, African, Hispanic, East Asian, Arab, etc. Note that, when the processing is executed at the timing when the subject takes the pareidolia test, the controller 11 may select a race based on a country of origin of a test-taker, etc. In this case, it becomes possible to generate a facial image according to the country of origin or race of the subject, and it becomes possible to generate a pareidolia test image suitable for a test subject.

The controller 11 generates a facial image based on the acquired original image and the selected race (S15). Specifically, the controller 11 sets the selected race in the decoder included in the generator of the facial image generation model M2, inputs the original image to the facial image generation model M2, and acquires a facial image (predicted image) output from the facial image generation model M2. Note that, when the facial image generation model M2 is prepared for each race, the controller 11 may select a race in step S14, then specifies the facial image generation model M2 corresponding to the selected race, and generate a facial image using the specified facial image generation model M2. In addition, when the facial image generation model M2 is configured to generate facial images for all set races without inputting racial information, the controller 11 may select a facial image for the selected race from facial images generated by the facial image generation model M2.

The controller 11 generates a pareidolia test image by synthesizing the noise image generated in step S12 with the facial image generated in step S15 (S16). For example, the controller 11 synthesizes the facial image at any position with the noise image. In the examples illustrated in FIG. 5A and FIG. 5B, the facial image is synthesized in the second quadrant (top right region) of the noise image. Note that a synthesis position of the facial image may be any position in a randomly selected quadrant, any position in a quadrant selected in a predetermined order, or a position designated by the user. In addition, the controller 11 calculates a ratio of black pixels in each quadrant or region of the noise image, and may set a quadrant or region in which the ratio of black pixels is less than a predetermined value as the synthesis position of the facial image, or set a white pixel region provided in advance in the noise image as the synthesis position of the facial image. Furthermore, the controller 11 may synthesize the facial image with the noise image after converting the synthesis position of the facial image into a white pixel.

The controller 11 stores the generated pareidolia test image in the storage unit 12 in association with the racial information (S17). The controller 11 performs processing of steps S11 to S12 for all seed images to be processed, performs processing of steps S13 to S15 for all original images to be processed, generates a pareidolia test image for each synthesis of the seed images and the original images, and stores the image for each race in the storage unit 12. In this way, it is possible to generate a pareidolia test image for each race, including a facial image having a facial feature of a person of each race, based on a seed image and an original image prepared in advance. Note that, as illustrated in FIG. 5A and FIG. 5B, a pareidolia test image including a facial image and a pareidolia test image including only a noise image without a facial image are used in the pareidolia test. Therefore, the server 10 generates a pareidolia test image including a facial image by the above-mentioned processing, and generates a pareidolia test image including only a noise image by performing only steps S11 to S12. In this way, it is possible to prepare a pareidolia test image that can be used in the pareidolia test.

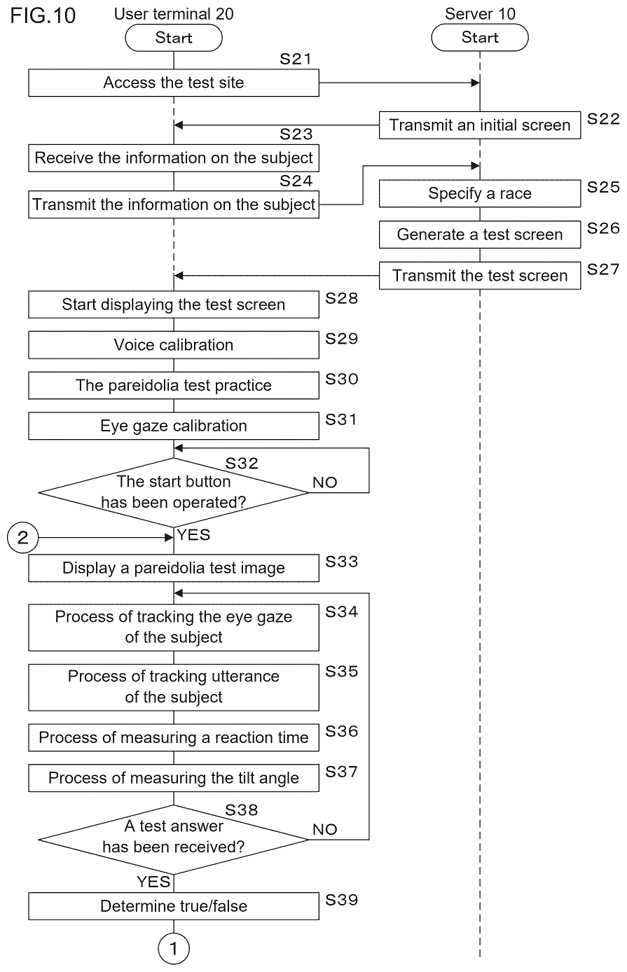

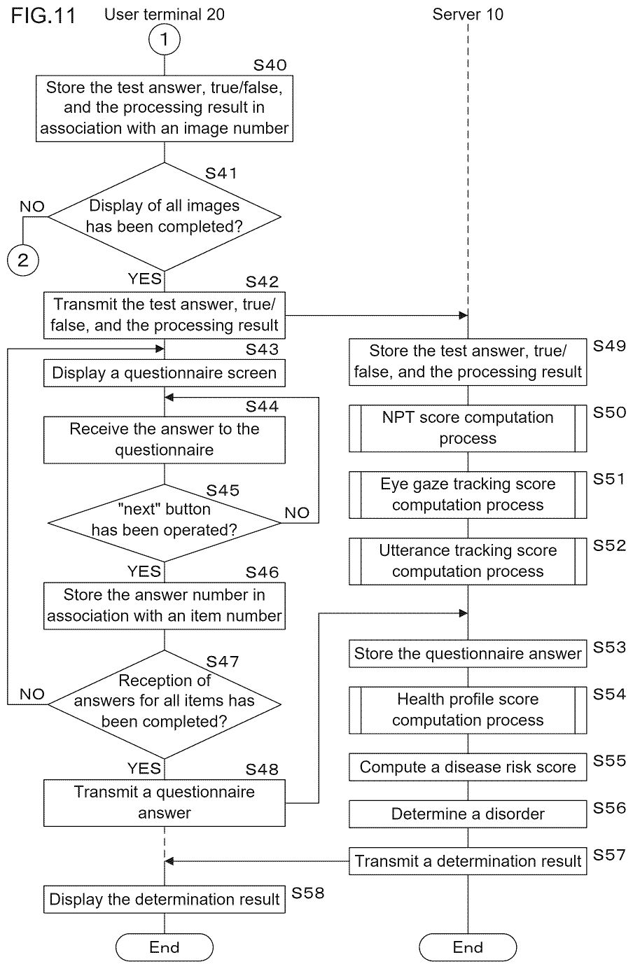

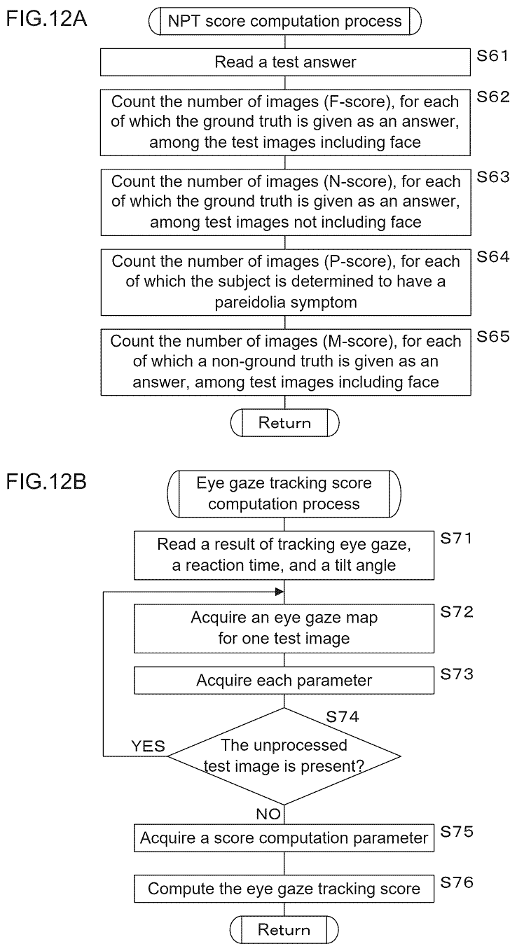

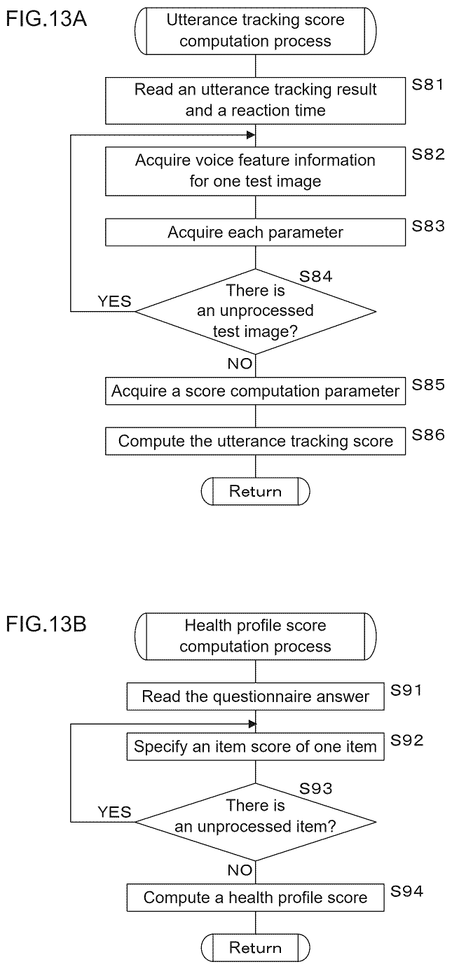

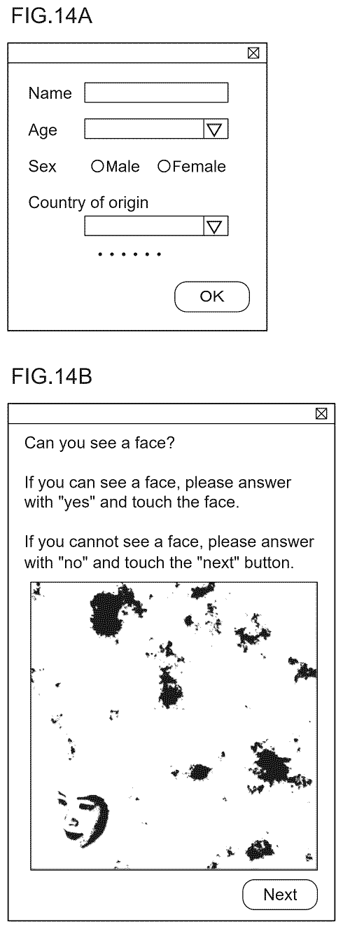

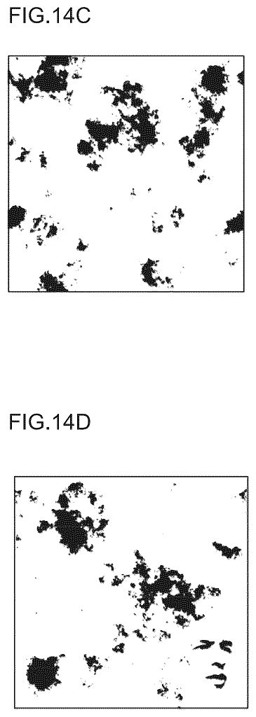

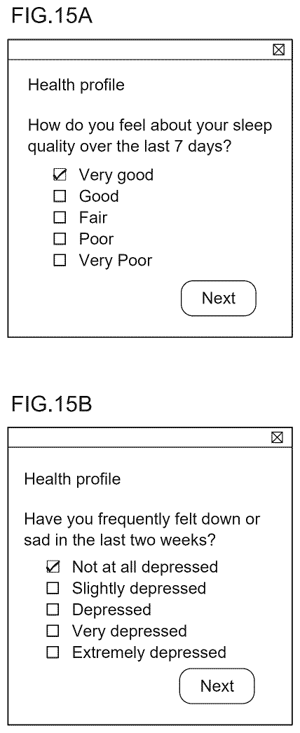

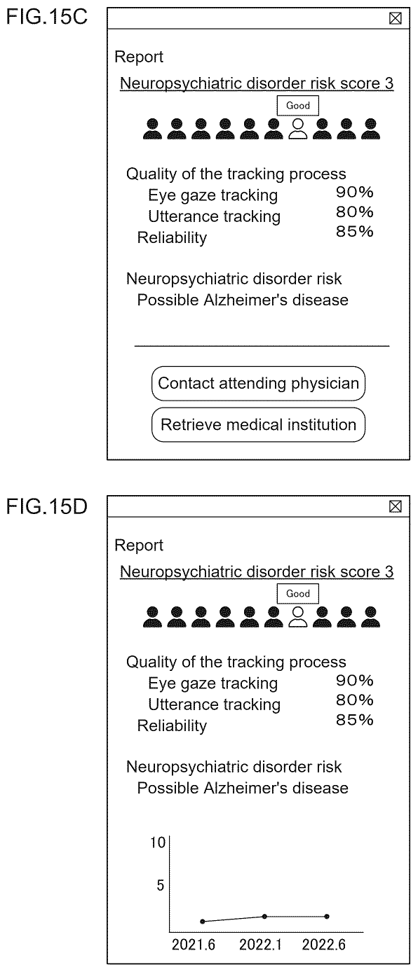

Hereinafter, a description will be given of processing of the pareidolia test in the information processing system of this embodiment. FIG. 10 to FIG. 13B are flowcharts each illustrating an example of a processing procedure of the pareidolia test, FIG. 14A to FIG. 15D are explanatory diagrams each illustrating a screen example of the user terminal 20, and FIG. 16A and FIG. 16B are explanatory diagrams of the pareidolia test of this embodiment. The following processing is executed by the controller 11 of the server 10 according to the program 12P stored in the storage unit 12, and executed by the controller 21 of the user terminal 20 according to the program 22P and test application 22AP stored in the storage unit 22. Note that an NPT score computation process illustrated in FIG. 12A is processing of step S50 of FIG. 11, an eye gaze tracking score computation process illustrated in FIG. 12B is processing of step S51 of FIG. 11, an utterance tracking score computation process illustrated in FIG. 13A is processing of step S52 of FIG. 11, and a health profile score computation process illustrated in FIG. 13B is processing of step S54 of FIG. 11.

A subject taking the pareidolia test accesses the test site 12S provided by the server 10 using the user terminal 20, and takes the pareidolia test according to information (screen) provided by the test site 12S. Upon receiving an instruction to execute the test application 22AP via the input unit 24, the controller 21 of the user terminal 20 starts the test application 22AP and accesses the test site 12S provided by the server 10 (S21). Upon receiving access to the test site 12S, the controller 11 of the server 10 transmits an initial screen to the user terminal 20 (S22). The controller 21 of the user terminal 20 displays the initial screen received from the server 10 on the display unit 25.

FIG. 14A illustrates an example of an initial screen, and the screen illustrated in FIG. 14A has input fields for a name, an age, a sex, a country of origin, etc. of the subject, and receives information related to the subject. The initial screen may have an input field for a race of the subject instead of a country of origin, and may further have input fields for a diagnostic name, a medical history, a medication history, etc. of the subject. Note that, when information on the subject is registered in advance in the server 10, the initial screen may have a configuration having an input field for identification information (for example, a user ID) assigned to the user. In addition, when the server 10 is managed by a medical institution, the initial screen may have a configuration having an input field for a patient card number of a patient card issued by the medical institution as identification information of the user. In this case, the server 10 can acquire the information on the subject from an electronic medical record system of the medical institution based on the patient card number acquired from the user terminal 20.

The controller 21 of the user terminal 20 receives the information on the subject via an input field on the initial screen (S23), and transmits the received information on the subject to the server 10 when an OK button is pressed (S24). Upon acquiring the information on the subject from the user terminal 20, the controller 11 of the server 10 specifies a race corresponding to the subject from among races prepared in advance (S25). For example, a table (not illustrated) in which each country is associated with a race that is predominant in each country may be stored in the storage unit 12, and the controller 11 may specify the race corresponding to the country of origin of the subject from the table. Note that, when an input field for a race is provided on the initial screen, the controller 11 may acquire the race from the user terminal 20 as the information on the subject. In addition, when a language in use is set for the test application 22AP, the user terminal 20 may transmit the language in use to the server 10 as the information on the subject, and in this case, the controller 11 may specify a race corresponding to the language in use. Note that, for example, the controller 11 can specify the race corresponding to the language in use using a table (not illustrated) in which each language is associated with a race that frequently uses the language.

The controller 11 generates a test screen for providing the subject with the pareidolia test according to the specified race (S26). Specifically, the controller 11 reads a pareidolia test image according to the race specified in step S25 from the storage unit 12. For example, the controller 11 reads, from the storage unit 12, a total of 40 pareidolia test images, namely, 8 pareidolia test images including a facial image of the specified race and 32 pareidolia test images including only noise images. Note that, when the storage unit 12 does not store a pareidolia test image including a facial image of each race, the controller 11 may generate a pareidolia test image including a facial image of a specified race at this point by performing processing of FIG. 9. The number of pareidolia test images is not limited to this example. The controller 11 rearranges the order of each pareidolia test image so that pareidolia test images including facial images are not consecutive, and generates a test screen on which one pareidolia test image is displayed per page.

The controller 11 (output unit) transmits (outputs) the generated test screen to the user terminal 20 (S27). The controller 21 of the user terminal 20 receives the test screen transmitted by the server 10, and starts displaying the received test screen on the display unit 25 (S28). A first screen (not illustrated) of the test screen displays a method of taking the pareidolia test, a caution, etc., and the user terminal 20 may output a precaution such as “If you normally wear glasses, please wear the glasses” as a voice message. In addition, the user terminal 20 of this embodiment is configured to perform calibration in an utterance tracking process and calibration in an eye gaze tracking process before starting the pareidolia test, thereby improving utterance tracking accuracy and eye gaze tracking accuracy, and as a result, improving test accuracy. Note that the user terminal 20 is configured to practice the pareidolia test by the subject after a calibration process in the utterance tracking process, and then to perform an actual pareidolia test after performing calibration in the eye gaze tracking process.

Therefore, the controller 21 executes calibration (voice calibration) in the utterance tracking process (S29). In voice calibration, for example, the controller 21 acquires voice of the subject reading aloud a given sentence using the microphone 27, and adjusts various parameters for converting a voice signal so that volume, frequency, etc. of the acquired voice fall within a preset reference range. In this way, it is possible to accurately acquire spoken voice of the subject. Note that voice calibration can be performed using general processing.

Next, the controller 21 executes a process related to practice of the pareidolia test (S30). For example, the controller 21 displays a test image for practice on the screen illustrated in FIG. 14B, and outputs a voice message such as “If there is a region appearing to be a face, answer with ”yes“ and touch the face. If there is no region appearing to be a face, answer with ”no“ and touch the ”next“ button”. The subject practices the pareidolia test according to an instruction of the user terminal 20. When the controller 21 acquires voice of “yes” uttered by the subject via the microphone 27 and receives a touch operation at any place on the test image via the input unit 24, the controller 21 receives an answer indicating that the place where the touch operation has been performed appears to be a face on the displayed pareidolia test image. In addition, when the controller 21 acquires voice of “no” uttered by the subject via the microphone 27 and receives a touch operation on the “next” button on the test image via the input unit 24, the controller 21 receives an answer indicating that there is no place where a face is seen on the displayed pareidolia test image. Upon receiving an answer from the subject, the controller 21 determines whether or not the answer is a ground truth based on whether a facial image is included in the displayed pareidolia test image and the answer from the subject, and displays a determination result. In this way, the subject can determine whether or not the answer from the subject is the ground truth. Note that the controller 21 may display a mark in a face region in the test image and present the test image to the subject, and in this case, the subject can detect the face region in the test image. The controller 21 is configured to perform practice using a plurality of test images, and when switching between test images, the controller 21 notifies the subject of switching of the test images by displaying a predetermined animation or outputting a predetermined beep sound.

Next, the controller 21 executes calibration (eye gaze calibration) in the eye gaze tracking process (S31). In eye gaze calibration, for example, the controller 21 displays a monochromatic background image on the display unit 25, moves a monochromatic circular image (tracking ball) on the background image, detects an eye gaze position (viewed position) of the subject at this time, and adjusts various parameters for converting the eye gaze position into a display position of the tracking ball so that the eye gaze position matches the display position. In this way, it is possible to accurately acquire the eye gaze position of the subject. Note that general processing can be used for eye gaze calibration. In this embodiment, by performing eye gaze calibration immediately before starting the actual pareidolia test, the subject can perform the actual test in the same posture as that during eye gaze calibration, and thus it becomes possible to perform the more accurate eye gaze tracking process during the pareidolia test. Note that voice calibration, practice of the pareidolia test, and eye gaze calibration are not limited to being performed in this order, and the order of execution of each process may be rearranged. Voice calibration and eye gaze calibration may be performed once when the pareidolia test is first performed, may be performed in response to an instruction from the subject, or may be performed each time the pareidolia test is performed. Note that, if calibration is performed when the pareidolia test is performed, it becomes possible to perform the pareidolia test with high accuracy.

When eye gaze calibration ends, for example, the controller 21 displays a start button to instructing the start of the actual pareidolia test. When the subject wishes to start the pareidolia test, the subject operates the start button. The controller 21 determines whether or not the start button has been operated (S32), and waits until the start button is operated upon determining that the start button has not been operated (S32: NO). Upon determining that the start button has been operated (S32: YES), the controller 21 switches display to a next screen (page) and displays a first pareidolia test image (S33). The screen illustrated in FIG. 14B is an example of the test screen on which the pareidolia test image is displayed, and the displayed pareidolia test image is configured to receive an operation on any position. The subject determines whether or not there is a region appearing to be a face in the displayed pareidolia test image, utters “yes” upon determining that the region is present, and operates the region appearing to be a face, thereby inputting an answer indicating that the operated position appears to be a face. The operation on the region appearing to be a face is performed as a touch operation when the input unit 24 is a touch panel and performed as a click operation when the input unit 24 is a mouse. In addition, upon determining that there is no region appearing to be a face, the subject utters “no”, and operates the “next” button”, thereby inputting an answer indicating that there is no region appearing to be a face. Note that, upon determining that there is a region appearing to be a face, the “next” button” may be operated after operating the region appearing to be a face.

The pareidolia test image illustrated in FIG. 14B includes a facial image of a person at the bottom left. However, a pareidolia test image not including a facial image may be displayed as illustrated in FIG. 14C, or a pareidolia test image including a facial image at the bottom right may be displayed as illustrated in FIG. 14D. In addition, even though the pareidolia test image of FIG. 14B includes a facial image of a Japanese person, a facial image according to the race of the subject may be included, and a facial image of a Caucasian person is included in the example illustrated in FIG. 14D. In addition, the example of FIG. 14B includes a facial image in which the eye gaze is directed in the left direction. However, as illustrated in FIG. 14D, a facial image in which the eye gaze is directed in the right direction may be included. Note that facial images included in the pareidolia test images (for example, eight pareidolia test images) used in one pareidolia test include facial images in which the eye gaze is directed in a plurality of directions such as the frontward direction, the leftward direction, the rightward direction, the upward direction, and the downward direction.

After displaying the pareidolia test image, the controller 21 performs a process of tracking the eye gaze of the subject (S34), a process of tracking utterance of the test subject (S35), a process of measuring a reaction time of the subject (S36), and a process of measuring the tilt angle of the user terminal 20 (S37). In the eye gaze tracking process of step S34, the controller 21 captures an image of the face of the subject visually recognizing the displayed pareidolia test image using the camera 26, performs a process of tracking movement of the eye gaze (eye movement) of the subject based on the captured image, and generates the eye gaze map as illustrated in FIG. 7A. The controller 21 detects a fixation point of the subject and a saccade moving between fixation points on the displayed pareidolia test image, and measures a fixation time at each fixation point. For example, the controller 21 expresses each position in the pareidolia test image by an XY coordinate system in which a top left point of the image is set to the origin, the rightward direction is set to an X-axis, and the downward direction is set to the Y-axis. Then, the controller 21 sets a place first viewed by the subject for a predetermined time (for example, 0.1 seconds) or more to a first fixation point, specifies a position of this first fixation point, and measures a fixation time. Next, upon detecting a saccade, the controller 21 sets a place subsequently viewed for the predetermined period of time or more to a second fixation point, specifies a position of this second fixation point, specifies a line segment from the first fixation point to the second fixation point as a trajectory of the saccade, and measures a fixation time of the second fixation point. The controller 21 continues this processing until the subject answers (an operation on a region appearing to be a face or an operation on the “next” button) via the input unit 24. In this way, the eye gaze map illustrated in FIG. 7A is generated, and it is possible to acquire the number of fixation points (number of fixations), an order, a position and a fixation time of each fixation point, the number of saccades, a movement time (saccade time), etc. from the eye gaze map. Note that, to generate an accurate eye gaze map from a captured image, the controller 11 may perform pre-processing to emphasize a part of a face region (a region of eyes) in the captured image so that features such as eye movement, an eye gaze pattern, blinking timing, and change in eye gaze (for example, a vertical direction, a horizontal direction, etc.) can be accurately extracted. The eye gaze tracking process for generating the eye gaze map may be performed by the server 10 in addition to the user terminal 20. In this case, the user terminal 20 may transmit an image captured by the camera 26 to the server 10.

In the utterance tracking process of step S35, the controller 21 acquires a voice uttered by the subject using the microphone 27, analyzes the spoken voice to perform a tracking process, and acquires voice feature information. The controller 21 detects a term uttered by the subject based on the acquired spoken voice, and counts the number of times of occurrence of each term. In addition, the controller 21 detects occurrence of spikes and peaks in the spoken voice, and acquires occurrence times, volumes, etc. of the detected spikes and peaks. In addition, the controller 21 acquires a fundamental frequency and a peak frequency of the spoken voice by performing, for example, a Fourier transform on the spoken voice. Note that the controller 21 can acquire highly accurate voice feature information by dividing a voice signal into segments for each term uttered by the subject and acquiring each feature for each segment. The controller 21 continues this processing until the subject answers via the input unit 24. In this way, for spoken voice corresponding to one pareidolia test image, the number of times of occurrence of each term is counted and voice feature information is acquired. Specifically, the controller 21 acquires the number of times of occurrence of spikes, the number of times of occurrence of peaks, the envelope peak, the fundamental frequency, the peak frequency, etc. In order to accurately acquire voice feature information from the spoken voice, the controller 11 may perform pre-processing such as a predetermined filtering process on the spoken voice so that the voice feature can be accurately extracted. The server 10 may perform the utterance tracking process for acquiring the voice feature information. In this case, the user terminal 20 may transmit the spoken voice acquired by the microphone 27 to the server 10.

In the process of measuring a reaction time of step S36, the controller 21 measures a reaction time from when one pareidolia test image is displayed until the subject answers. The answer from the subject may be when a touch operation is performed on a region appearing to the face, when the “next” button is operated, or when “yes” or “no” is uttered. In addition, the controller 21 may calculate, as the reaction time, an average value of reaction times for each pareidolia test image. In the process of measuring the tilt angle of step S37, the controller 21 measures the tilt angle of the user terminal 20 using the acceleration sensor 28. The controller 21 may calculate an average value of tilt angles measured during display of one pareidolia test image, or may specify a maximum value and a minimum value.

Upon receiving an operation on any place in the pareidolia test image on the test screen, the controller 21 receives a test answer from the subject indicating that there is a region appearing to be a face. On the other hand, upon receiving an operation on the “next” button without an operation on the pareidolia test image being performed, the controller 21 receives a test answer from the subject indicating that there is no region appearing to be a face. The controller 21 determines whether or not a test answer by the subject has been received (S38), and upon determining that a test answer has not been received (S38: NO), the controller 21 returns to step S34 and continues processing of steps S34 to S37 on the displayed pareidolia test image. Upon determining that a test answer has been received (S38: YES), the controller 21 determines whether or not the received test answer is the ground truth (true/false) (S39).

Each pareidolia test image includes information indicating whether or not the image includes a facial image, and when the facial image is included, information on a position of the facial image is included. Therefore, upon receiving an answer indicating that there is no region appearing to be a face for the test image including the facial image, the controller 21 determines that the answer is not the ground truth. On the other hand, upon receiving an answer indicating that there is a region appearing to be a face, it is determined whether the region answered by the subject is truly a region of the facial image. In the case of a correct facial image region, the answer is determined to be the ground truth, and in the case of an incorrect facial image region, the answer is determined not to be the ground truth and the subject is determined to have a pareidolia symptom. That is, even if the subject answers that there is a region appearing to be a face in the test image having a face image, when the subject answers that a place, which is not a facial image region, is a region appearing to be a face, the subject is determined to have a pareidolia symptom. In addition, for the test image not including a facial image, upon receiving an answer that there is no region appearing to be a face, the controller 21 determines that the answer is the ground truth, and upon receiving an answer that there is a region appearing to be a face, the controller 21 determines that the answer is not the ground truth and the subject has a pareidolia symptom.

The controller 21 stores the received test answer, a result of determination as to whether or not the answer is the ground truth, and each processing result of processing of steps S34 to S37 in the storage unit 22 in association with an image number of the displayed pareidolia test image (S40). The controller 21 determines whether display of all pareidolia test images has been completed on the test screen received from the server 10 (S41), and upon determining that display has not been completed (S41: NO), the controller 21 returns to processing of step S33, switches display to a next screen (page), and displays a next pareidolia test image (S33). Then, the controller 21 executes processing of steps S34 to S40 for the newly displayed pareidolia test image. In this way, for each pareidolia test image, the controller 21 can acquire the test answer, true/false of the answer, and processing results including the eye gaze map, the voice feature information, the reaction time, and the tilt angle of the user terminal 20. Note that, when switching display to the next screen, the controller 21 may notify the subject of switching of the test image by displaying a predetermined animation on the display unit 25 or outputting a predetermined beep sound from a speaker (not illustrated). In this way, the subject can detect that display of the test image has been switched, and allow the pareidolia test to smoothly proceed.

Upon determining that display of all the pareidolia test images has been completed (S41: YES), the controller 21 transmits the test answer, true/false of the answer, and the processing results stored in association with the image number to the server 10 (S42). Note that the controller 21 may transmit information indicating whether each pareidolia test image is an image including a facial image in association with the image number to the server 10. The controller 11 of the server 10 receives the test answer, true/false of the answer, and the processing results transmitted from the user terminal 20, and stores the test answer, true/false of the answer, and the processing results in the storage unit 12 in association with the image number (S49). In this way, the controller 11 (acquisition unit) can acquire information related to a response (answer) of the subject to the pareidolia test image. Specifically, the controller 11 acquires a visual recognition result (test answer) of the subject, an eye gaze map, and voice feature information.

After processing of step S42, the controller 21 of the user terminal 20 displays a questionnaire screen for the health profile on the display unit 25 (S43). Note that the questionnaire screen may be included in the test screen transmitted by the server 10 to the user terminal 20 in step S27, or may be received by the user terminal 20 from the server 10 at this point. The screens illustrated in FIG. 15A and FIG. 15B are examples of the questionnaire screen, and a questionnaire (question) related to one item and answer options are displayed on one page of the questionnaire screen. In the examples of FIG. 15A and FIG. 15B, five levels of options are displayed, but the disclosure is not limited to this configuration. The subject selects an answer that matches a question on the questionnaire screen and operates the “next” button. Note that content of the questionnaire may include, for example, whether or not the subject uses glasses or contact lenses, whether or not the subject has been diagnosed with a neurological disease, a current level of anxiety, a level of apathy and lethargy in the past week, etc. The controller 21 receives the answer to the questionnaire by the subject (S44), and determines whether or not the “next” button has been operated (S45). Upon determining that the button has not been operated (S45: NO), the controller 21 returns to processing of step S44 and continues to receive answers to the questionnaire. Upon determining that the “next” button has been operated (S45: YES), the controller 21 stores the received answer number (answer content) in the storage unit 22 in association with an item number of the questionnaire (S46).

The controller 21 determines whether or not reception of questionnaire answers for all items has been completed (S47), and upon determining that reception has not been completed (S47: NO), the controller 21 returns to processing of step S43, switches display to a next screen (page), and displays a questionnaire screen for a next item (S43). Then, the controller 21 executes processing of steps S44 to S46 for the newly displayed questionnaire screen. In this way, the controller 21 acquires an answer from the subject to each questionnaire item. Note that questionnaire items include an item related to an anxiety status, an item related to apathy, an item related to a diagnosed neurological disease, etc. in addition to an item related to sleep quality illustrated in FIG. 15A and an item related to a depressive status illustrated in FIG. 15B. Upon determining that reception of questionnaire answers of all items has been completed (S47: YES), the controller 21 transmits a stored questionnaire answer associated with an item number of a questionnaire to the server 10 (S48). The controller 11 of the server 10 receives a questionnaire answer transmitted from the user terminal 20, and stores the questionnaire answer in the storage unit 12 associated with the item number of the questionnaire (S53).

Meanwhile, after processing of step S49, the controller 11 of the server 10 computes an NPT score based on the test answer received from the user terminal 20 and true/false of the answer (S50). That is, the controller 11 computes a score of a pareidolia test similar to a conventional one based on a visual recognition result of the subject for the pareidolia test image. In the NPT score computation process illustrated in FIG. 12A, the controller 11 reads a test answer associated with an image number and true/false of the answer from the storage unit 12 (S61). The controller 11 counts the number of images, for each of which the ground truth is given as an answer, among the pareidolia test images including facial images (S62). An image for which the ground truth is given is an image, for which a region given as an answer from the subject as a region appearing to be a face is a correct facial image region. The number of images here is referred to as an F (Face)-score. In addition, the controller 11 counts the number of images, for each of which the ground truth is given as an answer (the number of images, for each of which an answer indicates that there is no region appearing to be a face), among pareidolia test images not including facial images (S63). The number of images here is referred to as an N (Noise)-score. In addition, the controller 11 counts the number of images, for each of which the subject is determined to have a pareidolia symptom (S64). Here, the controller 11 counts the sum of the number of images, for each of which a non-ground truth is given as an answer (the number of images, for each of which an answer indicates that there is a region appearing to be a face), among pareidolia test images not including facial images, and the number of images, for each of which a region given as an answer from the subject as a region appearing to be a face is not a correct facial image region, among pareidolia test images including facial images. The number of images here is referred to as a P (Pareidolia)-score. Furthermore, the controller 11 counts the number of images, for each of which a non-ground truth is given as an answer (the number of images, for each of which an answer indicates that there is no region appearing to be a face), among pareidolia test images including facial images (S65). The number of images here is referred to as an M (Missing image)-score. Upon computing the above-mentioned four scores based on test answers of the subject, the controller 11 returns to processing of FIG. 11.

Returning to processing of FIG. 11, the controller 11 computes an eye gaze tracking score based on a processing result of the eye gaze tracking process received from the user terminal 20 (S51). In the process of computing the eye gaze tracking score illustrated in FIG. 12B, the controller 11 reads a result of tracking eye gaze associated with each image number, a result of measuring a reaction time, and a result of measuring a tilt angle from the storage unit 12 (S71). Then, the controller 11 acquires an eye gaze map for one pareidolia test image (S72), and acquires each parameter indicating the result of tracking eye gaze based on the eye gaze map (S73). For example, the controller 11 acquires the number of fixations, and a fixation time and a quadrant of a first fixation point. In addition, the controller 11 acquires a fixation time of each fixation point, and computes the total fixation time. In addition, the controller 11 acquires the number of saccades, and computes the total time of each saccade (saccade time). In addition, the controller 11 divides each fixation point and each saccade for each quadrant, computes the sum (visit time) of the fixation time and the saccade time for each quadrant, and counts the sum (visit time) of the number of fixations and the number of saccades.

The controller 11 determines whether or not there is an unprocessed pareidolia test image for which each parameter of the result of tracking eye gaze has not been acquired (S74). Upon determining that the unprocessed pareidolia test image is present (S74: YES), the controller 11 returns to processing of step S72 and executes processing of steps S72 to S73 for the unprocessed pareidolia test image. In this way, the controller 11 acquires the above-mentioned parameter for each pareidolia test image. Upon determining that the unprocessed pareidolia test image is not present (S74: NO), the controller 11 acquires a parameter (score computation parameter) for computing an eye gaze tracking score based on each parameter acquired for each pareidolia test image (S75). The score computation parameter may be a parameter acquired for each pareidolia test image, a parameter acquired for some of the pareidolia test images, or an average value of each parameter. In addition, the controller 11 may acquire a reaction time for each pareidolia test image or an average value of the reaction times as the score computation parameter, and compute a tilt angle for each pareidolia test image or an average value of the tilt angle.

Then, the controller 11 computes each score of the eye gaze tracking score based on the score computation parameter (S76). Specifically, the controller 11 inputs the score computation parameter to the eye gaze tracking score computation model M3, and acquires each score output from the eye gaze tracking score computation model M3. Upon computing the eye gaze tracking score, the controller 11 returns to processing of FIG. 11.

Returning to processing of FIG. 11, the controller 11 computes an utterance tracking score based on a processing result of the utterance tracking process received from the user terminal 20 (S52). In the utterance tracking score computation process illustrated in FIG. 13A, the controller 11 reads an utterance tracking result associated with each image number and a reaction time measurement result from the storage unit 12 (S81). Then, the controller 11 acquires voice feature information for one pareidolia test image (S82), and acquires each parameter indicating an utterance tracking result for one pareidolia test image (S83). For example, the controller 11 computes a ratio (term occurrence frequency) of the number of times of occurrence of “yes” and “no” to the number of times of occurrence of all terms based on the number of times of occurrence of each term uttered by the subject. In addition, the controller 11 acquires the number of spikes, the number of peaks, the fundamental frequency, and the peak frequency, and generates an envelope peak indicating an average movement line of the peaks.

The controller 11 determines whether or not there is an unprocessed pareidolia test image for which each parameter of the utterance tracking result has not been acquired (S84). Upon determining that the unprocessed pareidolia test image is present (S84: YES), the controller 11 returns to processing of step S82 and executes processing of steps S82 to S83 for the unprocessed pareidolia test image. In this way, the controller 11 acquires the above-mentioned parameter for each pareidolia test image. Upon determining that there is no unprocessed pareidolia test image (S84: NO), the controller 11 acquires a parameter (score computation parameter) for computing an utterance tracking score based on each parameter acquired for each pareidolia test image (S85). The score computation parameter may be a parameter acquired for each pareidolia test image, a parameter acquired for some of the pareidolia test images, or an average value of each parameter. In addition, the controller 11 acquires a reaction time for each pareidolia test image or an average value of the reaction time as the score computation parameter. Note that the controller 11 may compute a tilt angle or an average value of the tilt angle for each pareidolia test image as the score computation parameter.

Then, the controller 11 computes each score of the utterance tracking score based on the score computation parameter (S86). Specifically, the controller 11 inputs the score computation parameter to the utterance tracking score computation model M4 and acquires each score output from the utterance tracking score computation model M4. Note that a voice calibration length input to the utterance tracking score computation model M4 is acquired, for example, in the user terminal 20 before the start of the pareidolia test, and is transmitted to the server 10 as the utterance tracking result. Upon computing the utterance tracking score, the controller 11 returns to processing of FIG. 11.

After processing of step S53, the controller 11 computes a health profile score based on the questionnaire answer received from the user terminal 20 (S54). In the health profile score computation process illustrated in FIG. 13B, the controller 11 reads the questionnaire answer associated with the item number from the storage unit 12 (S91). Then, the controller 11 specifies an item score of one item based on a questionnaire answer for the item (S92). For each item in the questionnaire, a score is set for each answer, and the controller 11 uses the score set for the questionnaire answer as an item score for the item. The controller 11 determines whether or not there is an unprocessed item for which an item score has not been determined (S93). Upon determining that the unprocessed item is present (S93: YES), the controller 11 returns to processing of step S92 and specifies an item score for the unprocessed item. Upon determining that the unprocessed item is not present (S93: NO), the controller 11 computes a health profile score based on the item score of each item (S94). Here, for example, the controller 11 may compute the health profile score by weighting and summing the item score of each item. For example, an item score related to sleep quality is more important than an item score related to apathy, and thus may be weighted more heavily. Note that weighting of each item score can be appropriately set and changed by an expert such as a doctor or researcher. Upon computing the health profile score, the controller 11 returns to processing of FIG. 11.

The controller 11 (computation unit) computes an NPT score, an eye gaze tracking score, an utterance tracking score, and a health profile score, and then computes a disease risk score (a risk score related to neuropsychiatric disorders) based on these scores (S55). Specifically, the controller 11 inputs the NPT score, each score of the eye gaze tracking score, each score of the utterance tracking score, and the health profile score to the disease risk score computation model M5, and acquires the disease risk score output from the disease risk score computation model M5. Note that, when the disease risk score computation model M5 is configured to receive input of information on the age, the sex, the medical history, the treatment history, the medication history, etc. of the subject in addition to the above-mentioned scores, the controller 11 inputs the above-mentioned scores and information on the subject acquired from the user terminal 20 to the disease risk score computation model M5, and acquires the disease risk score from the disease risk score computation model M5. For example, the disease risk score is expressed as a value from 0 to 10.

The controller 11 determines (specifies) a possible neuropsychiatric disorder among a plurality of neuropsychiatric disorders subjected to determination based on the computed disease risk score (S56). For example, it is preset that a disease risk score of less than 3 indicates a healthy state (a state without neuropsychiatric disorders), a disease risk score of 3 or more and less than 5 indicates Alzheimer's disease, a disease risk score of 5 or more and less than 7 indicates Parkinson's disease, and a disease risk score of 7 or more indicates Lewy body dementia or schizophrenia, and the controller 11 specifies a neuropsychiatric disorder according to the disease risk score. Note that a neuropsychiatric disorder specified by the disease risk score is not limited to the above-mentioned diseases, and a disease risk score set for each disease can be changed as appropriate. FIG. 16A illustrates disease risk scores obtained by carrying out the pareidolia test of this embodiment on healthy elderly people (for example, 65 years or older), and patients having respective diseases such as Alzheimer's disease, Parkinson's disease, Lewy body dementia, and schizophrenia. FIG. 16A illustrates a minimum value, a maximum value, and an average value (diamond) of disease risk scores of subjects for each disease. As illustrated in FIG. 16A, disease risk scores that can be taken by subjects are in different ranges depending on the disease, and thus it is possible to distinguish each disease by a disease risk score.

Note that FIG. 16B illustrates NPT scores computed for healthy elderly people, Parkinson's disease patients not having pareidolia symptoms, and Parkinson's disease patients having pareidolia symptoms. In FIG. 16B, a variation of NPT scores of subjects for each disease is illustrated as a box-and-whisker plot. The box-and-whisker plot of FIG. 16B expresses a minimum value, a first quartile, a median, a third quartile, and a maximum value of the NPT scores computed for the respective subjects using boxes and whiskers. As illustrated in FIG. 16B, in the NPT score, even when a patient has the same Parkinson's disease, if the patient does not have a pareidolia symptom, an NPT score similar to that of a healthy person is obtained. Since the NPT score is a score for determining the presence or absence of the pareidolia symptom, the presence or absence of a neuropsychiatric disorder such as Parkinson's disease cannot be determined only by the NPT score. On the other hand, in this embodiment, the presence or absence of a neuropsychiatric disorder can be determined using a disease risk score that takes into account a result of tracking eye gaze and a result of tracking utterance of the subject during the pareidolia test, as well as a health profile score, in addition to the NPT score. Therefore, it is possible to specify a patient having Parkinson's disease regardless of the presence or absence of a pareidolia symptom, and appropriate diagnosis becomes possible.

Note that neuropsychiatric disorders specified by the disease risk scores can be diseases that cause visual hallucinations, pareidolia, etc. For example, some or all of diseases such as front-temporal dementia, depressive disorder with psychosis, bipolar disorder with psychosis, alcohol withdrawal delirium, stimulant delirium due to cocaine or methamphetamine, posterior cortical atrophy, seizures, epilepsy, migraines, tumors, sleep disturbances, narcolepsy, stroke, peduncular hallucinosis, inborn errors in metabolism, Gerstmann syndrome, Creutzfeldt-Jakob disease, Charles Bonnet syndrome, and Anton's syndrome can be specified by disease risk scores. In addition, effects of hallucinogenic drugs including mescaline, psilocybin, lysergic acid diethylamide (LSD), phencyclidine (PCP), ecstasy, atropine, and dopamine agonists may be specified by disease risk scores.

When a disease risk score computed in the past is stored in the test application 22 AP or the server 10, in step S56 the controller 11 may determine (specify) possible neuropsychiatric disorders based on the time-series changes between the past disease risk score and the current disease risk score computed in step S55. In this case, by registering a state of time-series changes in the disease risk score in association with each disease, it is possible to determine (specify) a disease according to the time-series changes in the disease risk score. After processing of step S56, the controller 11 transmits a determination result to the user terminal 20 (S57), and the controller 21 of the user terminal 20 receives the determination result transmitted by the server 10 and displays the determination result on the display unit 25 (S58). For example, the controller 11 generates a report screen illustrated in FIG. 15C, and transmits the report screen to the user terminal 20. The report screen as illustrated in FIG. 15C displays a computed disease risk score (“3” in FIG. 15C), a determination result according to the disease risk score (“Good” in FIG. 15C), and a risk of a neuropsychiatric disorder determined based on the disease risk score (“possible Alzheimer's disease” in FIG. 15C).

In addition, for example, when information such as a contact address of an attending physician of the subject can be registered in the test application 22AP, the report screen may have a “contact attending physician” button for issuing an instruction to contact the attending physician of the subject. Furthermore, the report screen may have a “retrieve medical institution” button for issuing an instruction to retrieve a medical institution. In such a case, when the subject desires to contact attending physician with the pareidolia test result presented on the screen of FIG. 15C, the subject operates the “contact attending physician” button, and when the subject desires to retrieve the medical institution, the subject operates the “retrieve medical institution” button. When the “contact attending physician” button is operated on the screen of FIG. 15C, the controller 21 of the user terminal 20 transmits the pareidolia test result to a terminal used by the attending physician. In this instance, the controller 21 may transmit each piece of information acquired during the pareidolia test to the terminal of the attending physician. Note that an e-mail and an application such as LINE® can be used to transmit the pareidolia test result. In addition, when the “retrieve medical institution” button is operated, for example, the controller 21 acquires a current position of the user terminal 20, retrieves a medical institution having a department of neurology, a department of psychiatry, a department of neuropsychiatry, a department of psychosomatic medicine, a department of neurological medicine, a specialized outpatient clinic for neuropsychiatric disorders, etc. from among medical institutions around the current position, and displays a retrieval result on the display unit 25 to provide the retrieval result to the subject. Note that the current position of the user terminal 20 can be detected based on a GPS (Global Positioning System) signal detected by a GPS sensor provided in the user terminal 20. The medical institution can be retrieved using a search engine (search server) provided on the network N. In this way, when there is an attending physician, a pareidolia test result can be provided and shared with the attending physician, and when there is no attending physician or family doctor, medical institutions and doctors available for consultation can be presented, and early consultation at a medical institution can be encouraged.

Note that, when the test application 22AP or the server 10 stores disease risk scores computed in the past, the controller 11 may generate a report screen displaying a graph indicating time-series changes in disease risk score as illustrated in FIG. 15D. In addition, the screen of FIG. 15C and the screen of FIG. 15D may be switched and displayed. In addition, the controller 11 may compute a possibility of onset of each disease based on a disease risk score corresponding to each neuropsychiatric disorder and a disease risk score of the subject, and generate a report screen displaying the possibility of onset of each disease. For example, the controller 11 may generate a report screen displaying a message such as “Parkinson's disease: 80%, Alzheimer's disease: 10%, and Lewy body dementia: 10%”.

By the above-mentioned processing, in this embodiment, a disease risk score can be predicted by taking into consideration the result of tracking eye gaze of the subject acquired during the pareidolia test, the utterance tracking result, and the result of the health profile questionnaire answered by the subject in addition to a result of the conventional pareidolia test, and a risk of neuropsychiatric disorders can be determined with high accuracy by such a disease risk score. Therefore, while the conventional pareidolia test has been used to determine the presence or absence of pareidolia symptoms, the pareidolia test of this embodiment can predict the risk of neuropsychiatric disorders in addition to the presence or absence of pareidolia symptoms and present the predicted risk to the subject. In this way, by taking the pareidolia test using the user terminal 20 of the subject, the subject can detect not only the presence or absence of pareidolia symptoms but also the risk of neuropsychiatric disorders. Even though visual hallucinations such as pareidolia frequently appear in neuropsychiatric disorders, this embodiment makes it possible to predict such disorders early and accurately, and by visiting a medical institution as necessary, neuropsychiatric disorders can be detected early. Note that, by using the disease risk score of this embodiment, it is possible to predict not only neuropsychiatric disorders such as Parkinson's disease but also neurological conditions such as brain tumors and strokes.

In this embodiment, the subject can detect time-series changes in the risk (disease risk score) of neuropsychiatric disorders by periodically taking the pareidolia test of this embodiment. In this case, the user terminal 20 or the server 10 may be configured to specify a possibility of onset or a degree of progression of neuropsychiatric disorders from changes in the risk score based on the risk score related to neuropsychiatric disorders acquired in time series. In such a configuration, in particular, a subject diagnosed with a neuropsychiatric disorder or a subject predicted to be at high risk of a neuropsychiatric disorder can objectively detect a degree of progression of the neuropsychiatric disorder and determine the timing for visiting a medical institution. In addition, by tracking changes in disease risk scores over a long term, experts such as doctors (for example, neurologists, psychiatrists, etc.) can develop appropriate treatment strategies, such as changing a type of prescribed medication and increasing or decreasing a dosage of each medication. In addition, for example, the test application 22AP or the server 10 may have a notification function for causing the subject to take a new pareidolia test when a predetermined period of time (for example, 3 months or 6 months) has passed since the subject last took the pareidolia test. In this case, the subject can regularly take the pareidolia test by taking the pareidolia test according to a notification.

In addition, in this embodiment, disease risk scores can be provided to healthy people (for example, elderly people) not having neuropsychiatric disorders. Visual misperception such as pareidolia can be used as an early biomarker for Parkinson's disease, Lewy body dementia, Alzheimer's disease, etc. Therefore, by carrying out the pareidolia test of this embodiment on a healthy person, a disease risk score can be provided, and the presence or absence of a symptom such as pareidolia can be predicted based on the disease risk score.

In this embodiment, the eye gaze tracking score computation model M3 automatically extracts features of the result of tracking eye gaze for the subject to predict the eye gaze tracking score. Therefore, it is possible to predict the eye gaze tracking score by generating an eye gaze map based on a facial image of the subject captured using the camera 26 of the user terminal 20. In addition, since the utterance tracking score computation model M4 automatically extracts features of the utterance tracking result for the subject to predict the utterance tracking score, it is possible to predict the utterance tracking score by acquiring voice feature information based on the spoken voice of the subject acquired using the microphone 27 of the user terminal 20. Furthermore, since the disease risk score computation model M5 automatically extracts features of input information such as the NPT score, the eye gaze tracking score, the utterance tracking score, and the health profile score to predict the eye gaze tracking score, it is possible to predict the disease risk score based on each score without performing complex calculation.

In addition, in this embodiment, since the pareidolia test can be performed using a pareidolia test image according to race, a pareidolia test image can be prepared for each subject, and a pareidolia test customized for each subject can be performed. Therefore, since the pareidolia test can be performed using a test image including a facial image having an atmosphere with which the subject is familiar, the risk of accidentally overlooking a facial image can be reduced and accuracy of the pareidolia test can be improved.

In this embodiment, respective computation processes of the NPT score, the eye gaze tracking score, the utterance tracking score, the health profile score, and the disease risk score are not limited to configurations performed by the server 10. For example, the user terminal 20 can be configured to locally perform the computation processes by downloading the eye gaze tracking score computation model M3, the utterance tracking score computation model M4, and the disease risk score computation model M5 to the user terminal 20. In this case, processing of steps S50 to S56 of FIG. 10 and FIG. 11 may be executed by the controller 21 of the user terminal 20, and the controller 21 may notify the subject of the computed disease risk score and the determined neuropsychiatric disorders by displaying the computed disease risk score and the determined neuropsychiatric disorders on the display unit 25. Even in such a configuration, the same processing as that in the above-described embodiment is possible, and the same effects can be obtained.

### Embodiment 2

A description will be given of an information processing system that can present results of a pareidolia test including recognized utterance content of the subject by the user terminal 20 recognizing the utterance content during implementation of the pareidolia test using the user terminal 20. The information processing system of this embodiment can be realized using devices 10 and 20 similar to those of the information processing system of Embodiment 1, and therefore a description of a configuration of each device will be omitted.

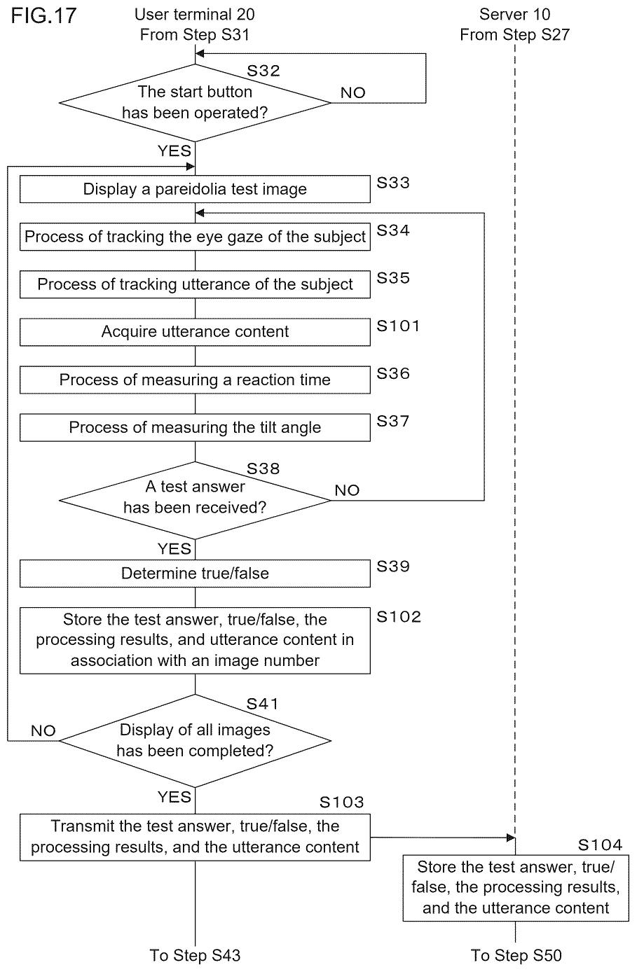

FIG. 17 is a flowchart illustrating an example of a processing procedure of a pareidolia test of Embodiment 2. Processing illustrated in FIG. 17 is obtained by adding step S101 between steps S35 and S36 and adding steps S102 to S104 instead of steps S40, S42, and S49, respectively, in processing illustrated in FIG. 10 and FIG. 11. A description of the same steps as those of FIG. 10 and FIG. 11 will be omitted. Note that illustration of steps S21 to S31, S43 to S48, and S50 to S58 of FIG. 10 and FIG. 11 is omitted in FIG. 17.

In this embodiment, the controller 21 of the user terminal 20 acquires utterance content uttered by the subject (S101) while performing an utterance tracking process based on spoken voice of the subject acquired by the microphone 27 in step S35. Note that the user terminal 20 has a voice input function via the microphone 27, and can acquire the utterance content from the spoken voice of the subject acquired by the microphone 27.

Then, after processing of step S39, the controller 21 stores the utterance content acquired in step S101 in the storage unit 22 in association with an image number of a pareidolia test image (S102), in addition to a test answer, true/false of the answer, and processing results of respective processes of steps S34 to S37. In addition, upon determining that display of all pareidolia test images has ended in step S41 (S41: YES), the controller 21 transmits the utterance content to the server 10 in addition to the test answer, true/false of the answer, and the processing results (S103). The controller 11 of the server 10 receives the test answer, true/false of the answer, the processing results, and the utterance content transmitted from the user terminal 20, and stores the test answer, true/false of the answer, the processing results, and the utterance content in the storage unit 12 in association with the image number (S104). In this way, the server 10 can acquire utterance content uttered by the subject during the pareidolia test, in addition to information related to the answer from the subject with respect to the pareidolia test image. Thereafter, the controller 11 executes processing from step S50 onwards.

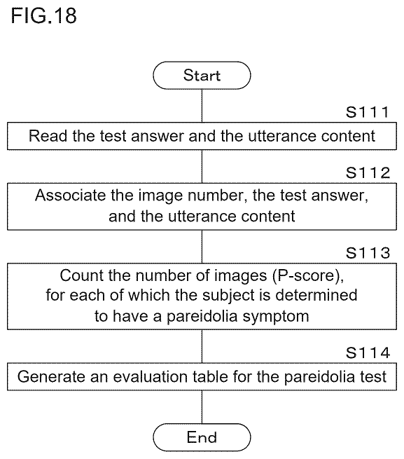

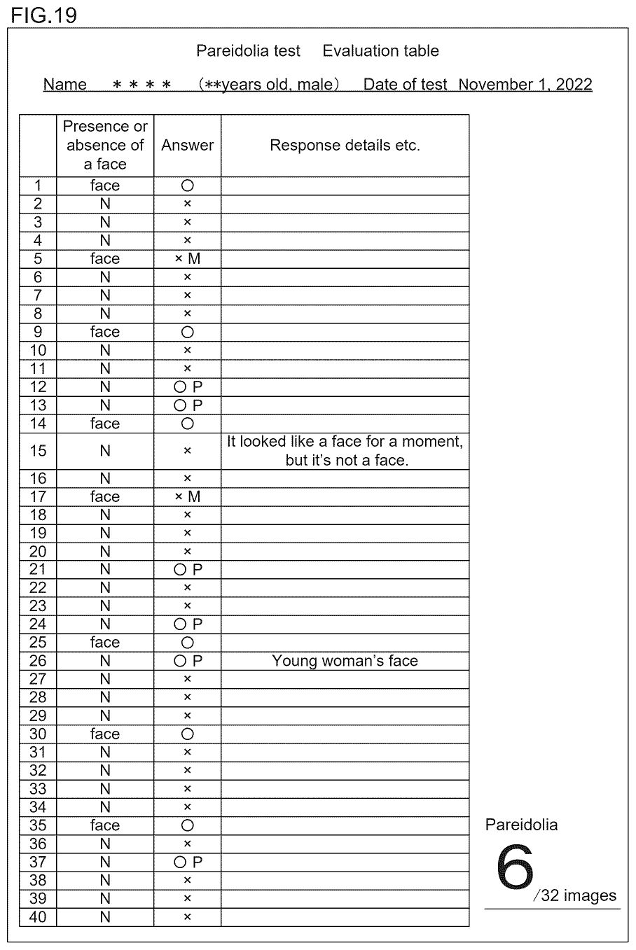

FIG. 18 is a flowchart illustrating an example of a processing procedure of generating an evaluation table for the pareidolia test, and FIG. 19 is an explanatory diagram illustrating an example of the evaluation table for the pareidolia test. After the subject has completed the pareidolia test, the controller 11 of the server 10 can generate the evaluation table for the pareidolia test by executing the following processing.

When generating an evaluation table for a pareidolia test for a certain subject, the controller 11 of the server 10 reads a test answer for the pareidolia test by the subject, true/false of the answer, and utterance content in the pareidolia test from the storage unit 12 (S111). The controller 11 associates an image number, the presence or absence of a facial image in the image, a test answer, and utterance content with each pareidolia test image (S112). In this instance, the controller 11 associates “pareidolia” as a test answer for a test image, for which the subject is determined to have a pareidolia symptom. In addition, when a test image including a facial image is not the ground truth, the controller 11 associates “missing image” as the test answer for the test image. The controller 11 counts the number of images, for each of which the subject is determined to have a pareidolia symptom (P-score) (S113).

Then, the controller 11 generates an evaluation table for the pareidolia test that displays a list of image numbers, the presence or absence of facial images, test answers, and utterance content associated in step S112 and displays P-scores computed in step S113 (S114). The evaluation table illustrated in FIG. 19 displays the presence or absence of a facial image (“face” when a facial image is included and “N” when a facial image is not included), a test answer (“O” when an answer indicates that there is a region appearing to be a face, “X” when an answer indicates that there is no region appearing to be a face, “P” when the subject is determined to have pareidolia, and “M” when an answer is not the ground truth for a test image including a facial image), and utterance content in each test image in association with each of image numbers from number 1 to number 40. In addition, the evaluation table illustrated in FIG. 19 indicates that, of 32 pareidolia test images not including facial images, the number of images, for each of which the subject is determined to have a pareidolia symptom, is six.

For example, the controller 11 may provide the subject with the evaluation table for the pareidolia test illustrated in FIG. 19 by transmitting the evaluation table to the user terminal 20 or print the evaluation table by transmitting the evaluation table to a printer with which the server 10 can communicate. In addition, when a contact address, etc. of the attending physician of the subject is registered in the server 10, the controller 11 may transmit the evaluation table for the pareidolia test to the terminal of the attending physician, etc. In this case, results of the pareidolia test taken by the subject can be shared with the attending physician.

In this embodiment, the user terminal 20 can recognize utterance content uttered by the subject during the pareidolia test, thereby adding the utterance content to the pareidolia test results. Therefore, as illustrated in FIG. 19, it is possible to automatically generate an evaluation table including not only the pareidolia test results but also utterance content uttered while visually recognizing each pareidolia test image. In this embodiment, similar effects to those of the above-mentioned Embodiment 1 are obtained. Moreover, modified examples described as appropriate in the above-mentioned Embodiment 1 can be applied to this embodiment.

### Embodiment 3

In this embodiment, a description will be given of an information processing system that computes a disease risk score by taking into account results of a short-term memory test in addition to the NPT score, the eye gaze tracking score, the utterance tracking score, and the health profile score. The information processing system of this embodiment can be realized using devices 10 and 20 similar to those of the information processing system of Embodiment 1, and therefore a description of a configuration of each device will be omitted.

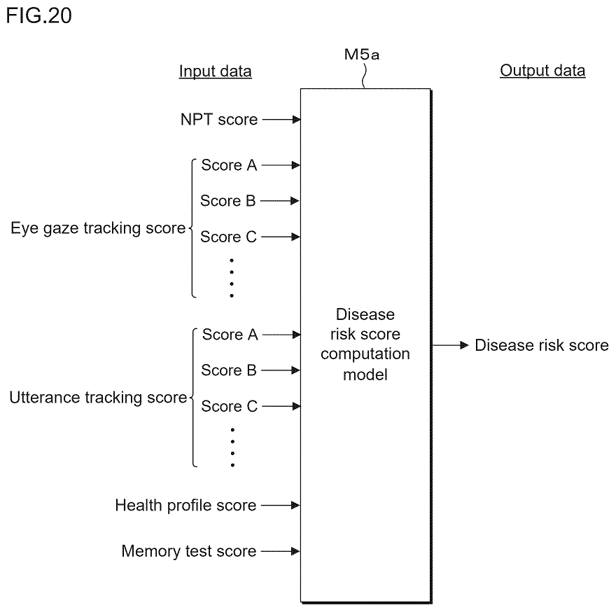

FIG. 20 is an explanatory diagram illustrating an overview of a disease risk score computation model M5a of Embodiment 3. The disease risk score computation model M5a illustrated in FIG. 20 has a similar configuration to that of the disease risk score computation model M5 of Embodiment 1 illustrated in FIG. 8. However, input data is different from that of the disease risk score computation model M5 of Embodiment 1. Specifically, in addition to an NPT score, an eye gaze tracking score, an utterance tracking score, and a health profile score, a memory test score indicating a result of a short-term memory test performed during a pareidolia test is input to the disease risk score computation model M5a. Therefore, the disease risk score computation model M5a performs computation to predict a disease risk score based on the memory test score in addition to the NPT score, the eye gaze tracking score, the utterance tracking score, and the health profile score, and outputs a computation result. In addition, the disease risk score computation model M5a can be generated by machine learning using training data including each training score including a memory test score and a disease risk score for a patient having the score.

The short-term memory test generally makes the subject to memorize three or more words, and then verifies whether or not the subject memorizes the words after a predetermined time has passed. The words used in the test are words used in daily life, and can be words from any category, such as colors, shapes, animals, and body parts. In addition, a method of presenting the words when making the subject memorize the words may be voice output, display on a monitor, or both of voice output and display on a monitor. In addition, the subject may select a word to be memorized from the presented words, and memorize the selected word. The short-term memory test of this embodiment includes an ultra-short memory test in which five words are output as voice and then immediately afterwards the subject is tested to determine whether or not the subject memorizes the words (can repeat the words) before the start of the pareidolia test, and a short-term memory test in which the subject is tested to determine whether or not the subject memorizes the words (recalls the words) after the end of the pareidolia test. Note that at least one of a score of the ultra-short memory test (repeat test) and the score of the short-term memory test (recall test) is input to the disease risk score computation model M5a of this embodiment.

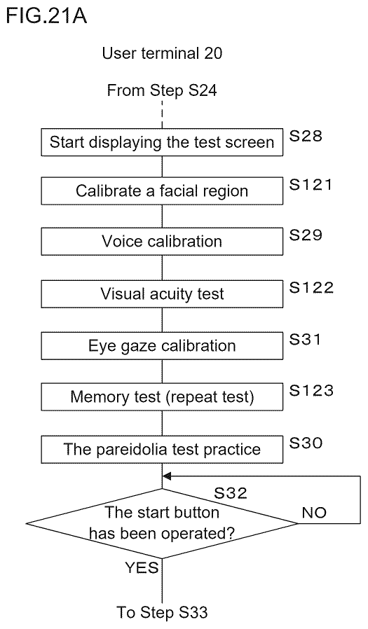

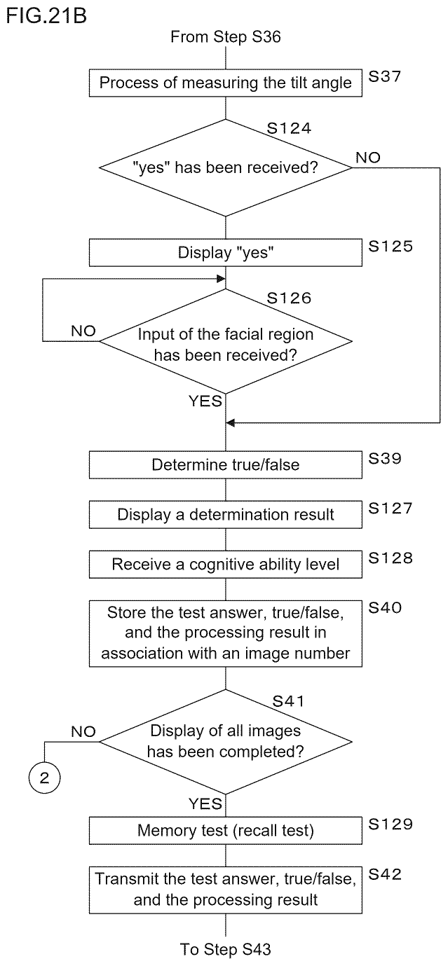

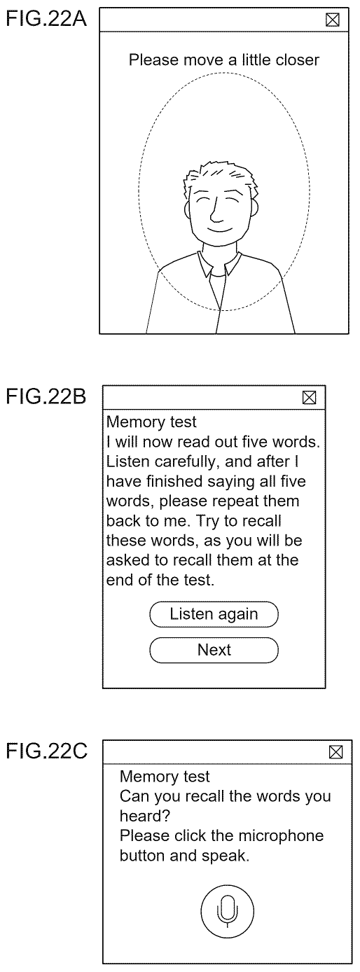

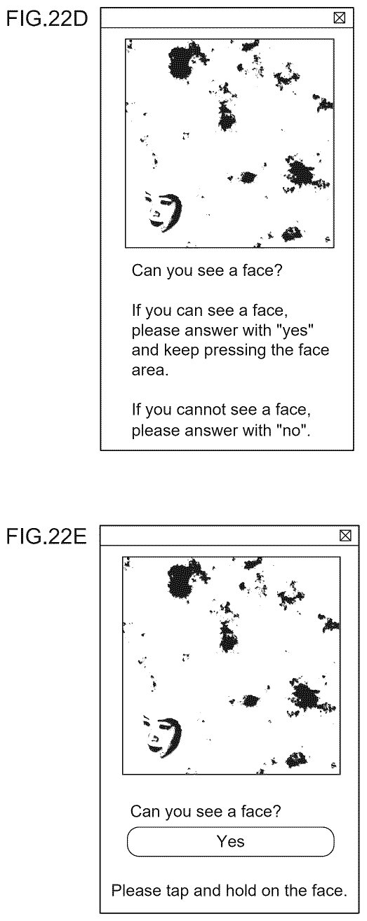

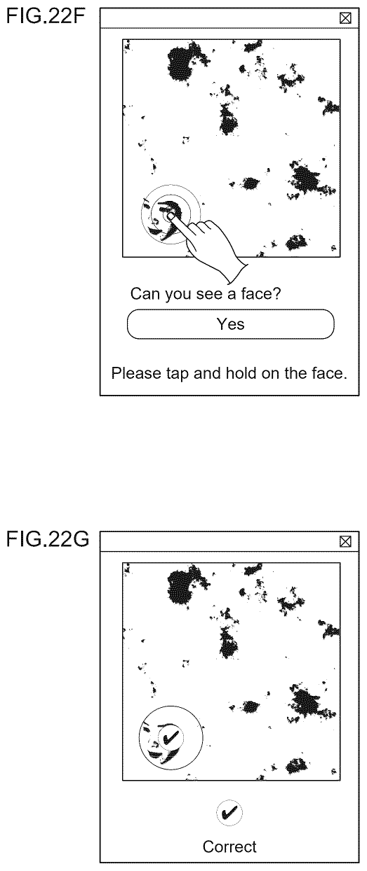

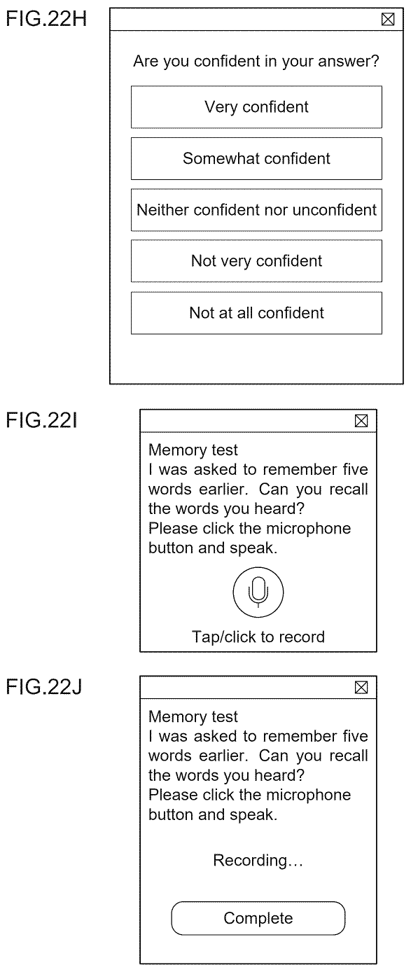

Hereinafter, a description will be given of processing of the pareidolia test in this embodiment. FIG. 21A and FIG. 21B are flowcharts illustrating examples of a processing procedure of the pareidolia test of Embodiment 3, and FIG. 22A to FIG. 22J are explanatory diagrams illustrating screen examples of the user terminal 20. Processing illustrated in FIG. 21A and FIG. 21B is obtained by adding step S121 between steps S28 and S29, moving step S30 to a position after step S31, adding step S122 between steps S29 and S31, adding step S123 between steps S31 and S30, adding steps S124 to S126 in place of step S38, adding steps S127 to S128 between steps S39 and S40, and adding step S129 between YES of step S41 and step S42 in processing illustrated in FIG. 10 and FIG. 11. A description of the same steps as those of FIG. 10 and FIG. 11 will be omitted. In FIG. 21A and FIG. 21B, illustration of steps S21 to S27, steps S33 to S36, and steps S43 to S58 of FIG. 10 and FIG. 11 is omitted.

In this embodiment, the controller 21 of the user terminal 20 starts display of a test screen for the pareidolia test in step S28, and then calibrates a facial region of the subject visually recognizing the test screen with respect to a capturing range of the camera 26 (S121). Here, as illustrated in FIG. 22A, the controller 21 displays a frame at a predetermined position in a display region of the display unit 25, and displays an image captured by the camera 26 on the display unit 25. In FIG. 22A, the frame indicated by a dashed line indicates a position where the facial region obtained by capturing the face of the subject needs to be displayed. The controller 21 executes face detection processing on the image captured by the camera 26 to detect the facial region in the captured image. Then, the controller 21 compares the detected facial region with a frame region, specifies a guidance message presenting an action that needs to be taken by the subject to make the facial region fit into the frame region, and outputs the guidance message. For example, when the facial region is small relative to the frame region, the controller 21 specifies a guidance message of “please move closer”, and when the facial region is large relative to the frame region, the controller 21 specifies a guidance message of “please move away”. In addition, when the facial region is shifted to the left relative to the frame region, the controller 21 specifies a guidance message of “please move left”, and when the facial region is shifted to the right relative to the frame region, the controller 21 specifies a guidance message of “please move right”. Note that the guidance message may be displayed on the display unit 25 or output as voice. The subject adjusts a positional relationship between the camera 26 (the user terminal 20) and the face of the subject in accordance with the guidance message so that the facial region of the subject enters the frame region. When the facial region of the subject fits the frame region, the controller 21 outputs a message notifying that calibration of the facial region has completed, and executes processing of step S29.

After processing of step S29, the controller 21 performs a visual acuity test on the subject (S122). Here, the controller 21 displays a visual acuity test screen on the display unit 25, on which a plurality of sizes of visual acuity test Landolt rings is displayed, and displays a message such as “please press the smallest one that shows the orientation of C”. Then, upon receiving an operation on any of the Landolt rings, the controller 21 outputs a message reporting that the visual acuity test has ended, and executes processing of step S31. Note that, when a size of the operated Landolt ring is equal to or larger than a predetermined size, for example, when the size is the largest size, the controller 21 may output a caution such as “if you normally wear glasses, please wear glasses”.

After processing of step S31, the controller 21 executes an ultra-short memory test (repeat test) (S123). Here, the controller 21 displays a screen illustrated in FIG. 22B on the display unit 25 and outputs a displayed message by voice. Thereafter, the controller 21 outputs words to be memorized (for example, five words, “face”, “silk”, “shrine”, “lily”, and “red”) by voice. Note that the controller 21 outputs each word by voice at a speed allowing an elderly person to hear and with a predetermined time interval (for example, one second) so that the subject can understand each word. In addition, the controller 21 may display text data of the words to be memorized on the display unit 25. When a “listen again” button on the screen of FIG. 22B is operated after five-word voice output ends, the controller 21 outputs the five words by voice again. When a “next” button on the screen of FIG. 22B is operated after five-word voice output ends, the controller 21 displays a screen illustrated in FIG. 22C and outputs a displayed message by voice. Then, when a microphone button on the screen is operated, the controller 21 starts collecting sound using the microphone 27 and acquires voice uttered (repeated) by the subject. The controller 21 converts the acquired voice data into text data, specifies words memorized (that could be repeated) by the subject among the words to be memorized based on the obtained text data, and counts the number of memorized words. The number of words counted here, that is, the number of words that could be repeated by the subject, becomes a score of the ultra-short memory test (repeat test score).

After specifying the repeat test score, the controller 21 stores the repeat test score in the storage unit 22 and executes processing related to practice of the pareidolia test (S30). In this embodiment, calibration in the facial region, voice calibration, visual acuity test, eye gaze calibration, ultra-short memory test, and practice of pareidolia test are not limited to being executed in this order, and the execution order of each process may be rearranged. Thereafter, the controller 21 executes processing of steps S32 to S37. Note that, in this embodiment, in step S33, the controller 21 displays a screen illustrated in FIG. 22D. On the screen of FIG. 22D, when there is a region appearing to be a face in a pareidolia test image, the subject utters “yes” and then performs a holding operation (long press) on the region appearing to be a face. In addition, upon determining that there is no region appearing to be a face, the subject utters “no”.

While performing processing of steps S34 to S37, the controller 21 determines whether or not voice input of “yes” by the subject has been received via the microphone 27 (S124), and upon determining that the voice input has been received (S124: YES), the controller 21 displays “yes” as illustrated in FIG. 22E (S125). In this way, the subject checks answer content of the subject from displayed content, and performs a holding operation on any place in the pareidolia test image, thereby inputting a region appearing to be a face. The controller 21 determines whether or not input of the region appearing to be a face has been received (S126). Here, first, the controller 21 determines whether or not the holding operation on any place in the pareidolia test image has been received, and upon determining that the holding operation has been received, the controller 21 displays a mark indicating the place on which the holding operation has been performed as illustrated in FIG. 22F. In FIG. 22F, the place on which the holding operation has been performed is indicated by a ripple-like animation. After checking the mark displayed on a screen of FIG. 22F, the subject ends the holding operation. When the holding operation ends, the controller 21 receives a region designated by the holding operation as a region appearing to be a face to the subject.

Upon determining that input of the region appearing to be a face to the subject has not been received (S126: NO), the controller 21 waits until the region is received. Upon determining that input of the region appearing to be a face to the subject has been received (S126: YES), the controller 21 determines whether or not the ground truth is obtained (true/false) for the region on which the holding operation has been performed (S39). Here, when the region on which the holding operation has been performed is a region of a correct facial image, the controller 21 determines that the ground truth is obtained, and when the region is not a region of a correct facial image, the controller 21 determines that the region is not the ground truth and the subject has a pareidolia symptom. Upon determining that voice input of “yes” by the subject has not been received (S124: NO), that is, upon determining that voice input of “no” by the subject has been received, the controller 21 proceeds to step S39, and determines whether or not the ground truth (true/false) is obtained for an answer indicating that there is no region appearing to be a face (S39). Here, when a region of a facial image is not included in the displayed pareidolia test image, the controller 21 determines that the ground truth is obtained, and when a region of a facial image is included therein, the controller 21 determines that the ground truth is not obtained.

The controller 21 displays a true/false determination result as illustrated in FIG. 22G (S127). A screen of FIG. 22G reports that a region on which the holding operation has been performed is a region of a facial image and an answer from the subject is the ground truth. Next, the controller 21 receives input of a cognitive ability level based on the presence or absence of confidence of the subject with respect to the answer (S128). Here, the controller 21 displays a screen illustrated in FIG. 22H. The screen of FIG. 22H has five buttons prepared, namely, “not at all confident”, “not very confident”, “neither confident nor unconfident”, “somewhat confident”, and “very confident”, and the controller 21 receives a degree of confidence with respect to the answer given to the pareidolia test image via each button. Note that, for example, the controller 11 receives a cognitive ability level 1 when receiving “not at all confident”, the controller 11 receives a cognitive ability level 2 when receiving “not very confident”, the controller 11 receives a cognitive ability level 3 when receiving “neither confident nor unconfident”, the controller 11 receives a cognitive ability level 4 when receiving “somewhat confident”, and the controller 11 receives a cognitive ability level 5 when receiving “very confident”. Thereafter, the controller 21 stores the received test result, the true/false determination result with respect to the answer, the cognitive ability level, and a processing results of each of processes of steps S34 to S37 in the storage unit 22 in association with an image number of the displayed pareidolia test image.

Upon determining that all the pareidolia test images have been displayed (S41: YES), the controller 21 of the user terminal 20 of this embodiment executes a short-term memory test (recall test) (S129). Here, the controller 21 displays a screen illustrated in FIG. 22I on the display unit 25 and outputs the displayed message by voice. Then, when a microphone button on the screen is operated, the controller 21 displays a screen illustrated in FIG. 22J and starts collecting sound by the microphone 27 to obtain voice uttered (recalled) by the subject. The controller 21 collects sound until a “complete” button on the screen of FIG. 22J is operated. The controller 21 converts acquired voice data into text data, specifies words memorized (that could be recalled) by the subject among the words to be memorized based on the obtained text data, and counts the number of memorized words. The number of words counted here, that is, the number of words that could be recalled by the subject, becomes a score of the short-term memory test (recall test score).

After specifying the recall test score, the controller 21 stores the recall test score in the storage unit 22. The controller 21 transmits the test answer, true/false of the answer, the cognitive ability level, and the processing results stored in association with the image number, and the memory test score (the repeat test score and the recall test score) to the server 10 (S42). Thereafter, the controller 21 executes processing from step S43 onward.

The controller 11 of the server 10 executes the same processing as that of processing of FIG. 10 and FIG. 11. Note that, in step S49, the controller 11 receives the test answer, true/false of the answer, the cognitive ability level, the processing results, and the memory test score transmitted from the user terminal 20, and stores the test answer, true/false of the answer, the cognitive ability level, the processing results, and the memory test score in the storage unit 12 in association with the image number. In this way, the server 10 of this embodiment can acquire the visual recognition result (the test answer, true/false of the answer, and the cognitive ability level) of the subject, the eye gaze map, the voice feature information, and the memory test result (the repeat test score and the recall test score) as information related to the response (answer) of the subject to the pareidolia test image. In addition, in step S55, the controller 21 inputs the memory test score (the repeat test score and the recall test score) received from the user terminal 20 in addition to the NPT score, the eye gaze tracking score, the utterance tracking score, and the health profile score to the disease risk score computation model M5a, and acquires a disease risk score output from the disease risk score computation model M5a.

In this embodiment, it is possible to acquire a disease risk score taking into account a result of the short-term memory test in addition to the NPT score, the eye gaze tracking score, the utterance tracking score, and the health profile score. Since short-term memory is affected by neuropsychiatric disorders, a more appropriate disease risk score can be acquired by taking into account the result of the short-term memory test. In addition, in this embodiment, not only the NPT score for the pareidolia test but also the cognitive ability level for each pareidolia test image can be acquired. Therefore, the disease risk score computation model M5a can be configured to receive input of the cognitive ability level for each pareidolia test image in addition to the above-mentioned score. In this case, the controller 11 may be configured to input the above-mentioned score and the cognitive ability level for each pareidolia test image to the disease risk score computation model M5a and acquire the disease risk score from the disease risk score computation model M5a. Note that, even in the above-mentioned Embodiment 1, when the controller 21 of the user terminal 20 is configured to receive input of the cognitive ability level for each pareidolia test image, the cognitive ability level may be included in input data of the disease risk score computation model M5.

In this embodiment, a process of computing each of the NPT score, the eye gaze tracking score, the utterance tracking score, the health profile score, and the disease risk score is not limited to a configuration performed by the server 10. By downloading some or all of the eye gaze tracking score computation model M3, the utterance tracking score computation model M4, and the disease risk score computation model M5 to the user terminal 20, the user terminal 20 can be configured to locally perform some or all of the processes of computing these scores. In such a configuration, processing similar to that of the above-mentioned embodiment is possible, and similar effects are obtained. In addition, when the user terminal 20 locally executes each process, there is no need to communicate with the server 10, and thus a processing time is shortened.

In this embodiment, the NPT score, the eye gaze tracking score, the utterance tracking score, the health profile score, and the memory test score are input to the disease risk score computation model M5a, and the disease risk score is acquired from the disease risk score computation model M5a. However, the disclosure is not limited to this configuration. For example, the disease risk score computation model M5 of each of Embodiments 1 and 2 may be used to acquire a disease risk score from the NPT score, the eye gaze tracking score, the utterance tracking score, and the health profile score, and determine a final disease risk score according to a combination of the acquired disease risk score and the memory test score.

In this embodiment, the cognitive ability level (the degree of confidence) of the subject is received for the answer to each pareidolia test image during the pareidolia test. Therefore, it is possible to predict the disease risk score by taking into account the cognitive ability level that the subject is conscious of in addition to the cognitive ability level computed from the result of tracking the spoken voice of the subject (the cognitive ability level that the subject is not aware of).

The configuration of this embodiment is applicable to the information processing system of each of the above-mentioned Embodiments 1 and 2, and even when the configuration is applied to the information processing system of each of the above-mentioned Embodiments 1 and 2, similar processing is possible, and similar effects are obtained. In addition, in this embodiment, modified examples described as appropriate in the above-mentioned Embodiments 1 and 2 can be applied to this embodiment. In addition, in each of the above-described embodiments, the disease risk score computed by the server 10 or the determination result according to the disease risk score does not have to be transmitted to the user terminal 20. For example, the server 10 may be configured to accumulate the disease risk score and the determination result, and provide the disease risk score or the determination result of each subject in response to a request from, for example, the attending physician of the subject, etc.

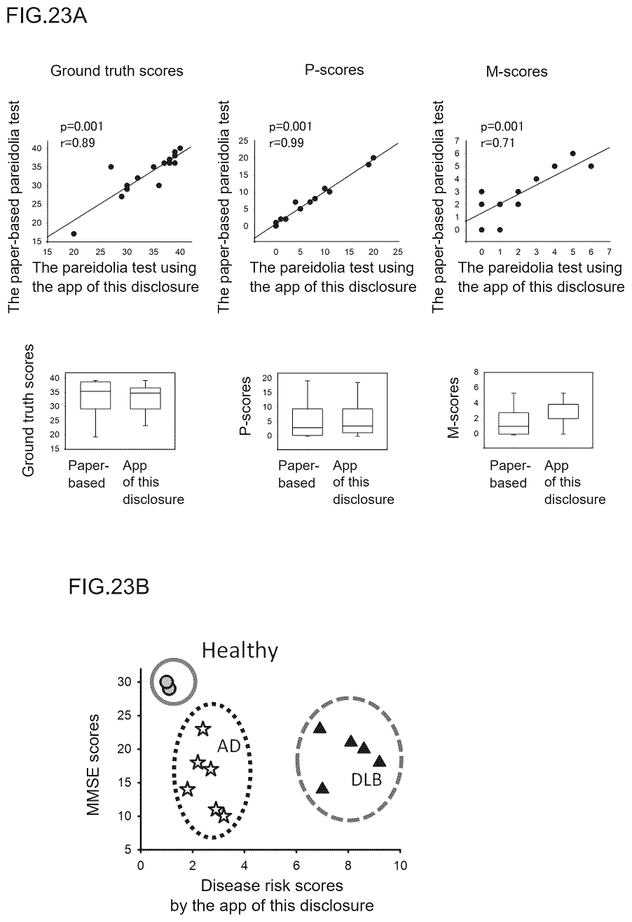

The inventors of this application verified validity of the disease risk score presented by the information processing system of the disclosure. FIG. 23A and FIG. 23B are explanatory diagrams illustrating verification results. The inventors of this application set, as subjects, four healthy people, six patients each having Alzheimer's disease, and five patients each having Lewy body dementia, conducted a pareidolia test using a pareidolia test image printed on paper (paper-based pareidolia test) and a pareidolia test using the test application 22AP (the application of the disclosure) installed in the user terminal 20 in the information processing system of the disclosure on each of the subjects, and compared respective test results. Note that subjects were aged 50 years or older, and disease duration of each of the patients having Alzheimer's disease or Lewy body dementia was 1 year or more.

FIG. 23A illustrates a result of comparison of NPT scores between the paper-based pareidolia test and the pareidolia test using the test application 22AP. A left side of FIG. 23A illustrates a result of comparison of ground truth scores in the pareidolia test. A ground truth score is the sum of the number of images (F-score), for which a region given as an answer from the subject as a region appearing to be a face is a correct facial image region, and the number of images (N-score), for each of which the subject gives an answer indicating that there is no region appearing to be a face, among pareidolia test images not including facial images. An upper left side illustrates a graph plotting, for each subject, the ground truth scores in the paper-based pareidolia test and the ground truth scores in the test using the test application 22AP, and a lower left side illustrates, as a box-and-whisker plot, a variation of ground truth scores of each subject for the paper-based ground truth scores and the ground truth scores in the test application 22AP. The box-and-whisker plot expresses a minimum value, a first quartile, a median, a third quartile, and a maximum value of the ground truth scores for each subject using boxes and whiskers.

A center of FIG. 23A illustrates a result of comparison of P (pareidolia)-scores in the pareidolia test. A P-score is the sum of the number of images, for each of which an answer indicates that there is a region appearing to be a face, among pareidolia test images not including facial images, and the number of images, for each of which a region given as an answer from the subject as a region appearing to be a face is not a correct facial image region, among pareidolia test images including facial images. An upper center side illustrates a graph plotting, for each subject, the P-scores in the paper-based pareidolia test and the P-scores in the test using the test application 22AP, and a lower center side illustrates, as a box-and-whisker plot, a variation of P-scores of each subject for the paper-based P-scores and the P-scores in the test application 22AP.

A right side of FIG. 23A illustrates a result of comparison of M (missing image)-scores in the pareidolia test. An M-score is the number of images, for each of which an answer indicates that there is no region appearing to be a face, among pareidolia test images including facial images. An upper right side illustrates a graph plotting, for each subject, the M-scores in the paper-based pareidolia test and the M-scores in the test using the test application 22AP, and a lower right side illustrates, as a box-and-whisker plot, a variation of M-scores of each subject for the paper-based M-scores and the M-scores in the test application 22AP.

From FIG. 23A, it can be seen that there is a high correlation between each score (ground truth score, P-score, and M-score) in the paper-based pareidolia test and each score in the pareidolia test using the test application 22AP. This indicates that the subject performs the same behavior in a paper-based test method and a test method using the user terminal 20 such as a smartphone, and it can be seen that there is no difference in the NPT score between the two methods. Therefore, the pareidolia test using the test application 22AP of the disclosure has the same effectiveness as that of the conventional paper-based pareidolia test.

Next, the inventor of this application performed Mini-Mental State Examination (MMSE), which is one of the dementia screening tests, to each of the subjects described above, and compared MMSE scores with disease risk scores obtained by the test application 22AP. FIG. 23B illustrates a graph plotting the MMSE scores and the disease risk scores obtained by the test application 22AP for each subject. As illustrated in FIG. 23B, using the MMSE scores, it is possible to distinguish whether a person is healthy or not, but it is impossible to distinguish between Alzheimer's disease (AD) patients and Lewy body dementia (DLB) patients. On the other hand, as illustrated in FIG. 23B, the test using the test application 22AP of the disclosure can distinguish between Alzheimer's disease patients and Lewy body dementia patients. By operating the test application 22AP in this way, it is possible to distinguish between Alzheimer's disease and Lewy body dementia, which enables early detection of the disease and allows early start of treatment according to a condition of a patient.

The respects described in the above embodiments can be combined with each other. In addition, the independent and dependent claims set forth in the claims can be combined with each other in any and all combinations, regardless of the format of reference. Further, the claims are in a format in which a claim refers to two or more other claims (the format of a multiple dependent claim), but are not limited thereto. The claims may be in a format in which a multiple dependent claim refers to at least one of multiple dependent claims (a multiple-multiple dependent claim).

It is to be noted that the disclosed embodiment is illustrative and not restrictive in all aspects. The scope of the present invention is defined by the appended claims rather than by the description preceding them, and all changes that fall within metes and bounds of the claims, or equivalence of such metes and bounds thereof are therefore intended to be embraced by the claims.

It is noted that, as used herein and in the appended claims, the singular forms “a”, “an”, and “the” include plural referents unless the context clearly dictates otherwise.

1-16. (canceled)

17. A non-transitory computer-readable storage medium storing a program causing a computer to execute processing of:
outputting a test image used in a pareidolia test;
acquiring information related to a response of a subject to the test image; and
computing a risk score related to a neuropsychiatric disorder based on the information related to the response of the subject.

18. The non-transitory computer-readable storage medium according to claim 17, wherein the program causes the computer to execute processing of:
acquiring a visual recognition result of the subject with respect to the test image;
computing a score of a pareidolia test based on the visual recognition result of the subject; and
computing the risk score related to the neuropsychiatric disorder based on the information related to the response of the subject including the score of the pareidolia test.

19. The non-transitory computer-readable storage medium according to claim 18, wherein the program causes the computer to execute processing of:
detecting an eye gaze of the subject with respect to the test image;
generating an eye gaze map indicating a fixation point and a saccade of the subject based on the eye gaze;
computing an eye gaze tracking score based on the eye gaze map; and
computing the risk score related to the neuropsychiatric disorder based on the information related to the response of the subject including the score of the pareidolia test and the eye gaze tracking score.

20. The non-transitory computer-readable storage medium according to claim 19, wherein the program causes the computer to execute processing of acquiring an eye gaze tracking score of the subject by inputting, to a learning model trained to output an eye gaze tracking score in response to input of information related to a fixation point and a saccade indicated by an eye gaze map, information related to the fixation point and the saccade of the subject indicated by the generated eye gaze map.

21. The non-transitory computer-readable storage medium according to claim 17, wherein the program causes the computer to execute processing of:
acquiring spoken voice of the subject visually recognizing the test image;
acquiring information related to a voice feature from the spoken voice;
computing an utterance tracking score based on the information related to the voice feature; and
computing the risk score related to the neuropsychiatric disorder based on the information related to the response of the subject including the score of the pareidolia test and the utterance tracking score.

22. The non-transitory computer-readable storage medium according to claim 21, wherein the program causes the computer to execute processing of acquiring the utterance tracking score of the subject by inputting, to a learning model trained to output an utterance tracking score in response to input of information related to a voice feature, the acquired information related to the voice feature.

23. The non-transitory computer-readable storage medium according to claim 17, wherein the program causes the computer to execute processing of:
acquiring an answer to a health profile questionnaire;
computing a health profile score based on the acquired answer; and
computing the risk score related to the neuropsychiatric disorder based on the information related to the response of the subject including the score of the pareidolia test and the health profile score.

24. The non-transitory computer-readable storage medium according to claim 21, wherein the program causes the computer to execute processing of:
acquiring an answer to a health profile questionnaire;
computing a health profile score based on the acquired answer; and
computing the risk score related to the neuropsychiatric disorder based on the information related to the reaction of the subject including the score of the pareidolia test, the utterance tracking score, and the health profile score.

25. The non-transitory computer-readable storage medium according to claim 17, wherein the program causes the computer to execute processing of:
outputting a plurality of words used in a memory test;
receiving input of the words at a predetermined timing;
computing a memory test score based on the received words; and
computing the risk score related to the neuropsychiatric disorder based on the information related to the reaction of the subject including the score of the pareidolia test and the memory test score.

26. The non-transitory computer-readable storage medium according to claim 17, wherein the program causes the computer to execute processing of:
generating a noise pattern image;
generating a facial image having an arbitrary eye gaze direction; and
generating the test image by synthesizing the generated noise pattern image and the generated facial image.

27. The non-transitory computer-readable storage medium according to claim 26, wherein:
the noise pattern image includes a noise pattern inducing pareidolia, and
the program causes the computer to execute processing of:
generating the noise pattern image from a seed image represented as a binary image using a random field model; and
inputting a binary facial image to a learning model trained to output a facial image of a different race from a race of a binary facial image in response to input of the facial image, thereby acquiring a facial image of a different race from a race of the input facial image.

28. The non-transitory computer-readable storage medium according to claim 17, wherein the program causes the computer to execute processing of specifying any one of a plurality of neuropsychiatric disorders subjected to determination based on the risk score related to the neuropsychiatric disorder.

29. The non-transitory computer-readable storage medium according to claim 28, wherein the program causes the computer to execute processing of outputting a determination result including the computed risk score related to the neuropsychiatric disorder or the specified neuropsychiatric disorder.

30. The non-transitory computer-readable storage medium according to claim 17, wherein the program causes the computer to execute processing of:
storing a risk score related to the neuropsychiatric disorder computed in time series; and
specifying a possibility of onset of the neuropsychiatric disorder based on changes in the risk score related to the neuropsychiatric disorder.

31. An information processing method in which a computer executes processing of:
outputting a test image used in a pareidolia test;
acquiring information related to a response of a subject to the test image; and
computing a risk score related to a neuropsychiatric disorder based on the information related to the response of the subject.

32. An information processing device including a controller,
wherein the controller is configured to:
output a test image used in a pareidolia test to a display unit;
acquire information related to a response of a subject to the test image; and
compute a risk score related to a neuropsychiatric disorder based on the information related to the response of the subject.
